SEQUENCE LISTING (26 sequences)

SEQ ID NO: 1 - DNA, 14 bp, Escherichia coli
cgtcaatgagcaaa

SEQ ID NO: 2 - DNA, 25 bp, Escherichia coli
cttctgcgggtaacgtcaatgagca

SEQ ID NO: 3 - DNA, 80 bp, Escherichia coli
gagtaaagttaatacctttgctcattgacgttacttacccgcagaagaagcaccggctaactccgtgccagcagccgcgg

SEQ ID NO: 4 - DNA, 80 bp, Escherichia coli
gagtaaagttaatacctttgcttattgacgttacttacccgcagaagaagcaccggctaactccgtgccagcagccgcgg

SEQ ID NO: 5 - DNA, 80 bp, Escherichia coli
gagtaaagttaatacctttgcttgttgacgttacttacccgcagaagaagcaccggctaactccgtgccagcagccgcgg

SEQ ID NO: 6 - DNA, 80 bp, Escherichia coli
gagtaaagttaatacctttgctggatgacgttacttacccgcagaagaagcaccggctaactccgtgccagcagccgcgg

SEQ ID NO: 7 - DNA, 80 bp, Escherichia coli
gagtaaagttaatacctttgcgacgtgacgttacttacccgcagaagaagcaccggctaactccgtgccagcagccgcgg

SEQ ID NO: 8 - DNA, 80 bp, Escherichia coli
gagtaaagttaatacctttgcgacgcgacgttacttacccgcagaagaagcaccggctaactccgtgccagcagccgcgg

SEQ ID NO: 9 - DNA, 80 bp, Escherichia coli
aggaagggagtaaagttaatacctttgctcattgacgttacccgcagaagaagcaccggctaactccgtgccagcagccg

SEQ ID NO: 10 - DNA, 80 bp, Escherichia coli
aggaagggagtaaagttaatacctttgctcattgacgatacccgcagaagaagcaccggctaactccgtgccagcagccg

SEQ ID NO: 11 - DNA, 80 bp, Escherichia coli
aggaagggagtaaagttaatacctttgctcattgacgagacccgcagaagaagcaccggctaactccgtgccagcagccg

SEQ ID NO: 12 - DNA, 80 bp, Escherichia coli
aggaagggagtaaagttaatacctttgctcattgactagacccgcagaagaagcaccggctaactccgtgccagcagccg

SEQ ID NO: 13 - DNA, 80 bp, Escherichia coli
aggaagggagtaaagttaatacctttgctcattgactagccccgcagaagaagcaccggctaactccgtgccagcagccg

SEQ ID NO: 14 - DNA, 80 bp, Escherichia coli
aggaagggagtaaagttaatacctttgctcattgattagccccgcagaagaagcaccggctaactccgtgccagcagccg

SEQ ID NO: 15 - DNA, 80 bp, Escherichia coli
ctcagagtatatacattccatagatctggatacccgtgacgaataaagatcagagtatatacattccatagatctgcata

SEQ ID NO: 16 - DNA, 80 bp, Escherichia coli
gagtaaagttaatacctttgctcattgacgttacccgcagaagaagcaccggctaactccgtgccagcagccgcggtaat

SEQ ID NO: 17 - DNA, 80 bp, Escherichia coli
gagtaaagttaatacctttgcttattgacgttacccgcagaagaagcaccggctaactccgtgccagcagccgcggtaat

SEQ ID NO: 18 - DNA, 80 bp, Escherichia coli
gagtaaagttaatacctttgcttgttgacgttacccgcagaagaagcaccggctaactccgtgccagcagccgcggtaat

SEQ ID NO: 19 - DNA, 80 bp, Escherichia coli
gagtaaagttaatacctttgctggatgacgttacccgcagaagaagcaccggctaactccgtgccagcagccgcggtaat

SEQ ID NO: 20 - DNA, 80 bp, Escherichia coli
gagtaaagttaatacctttgcgacgtgacgttacccgcagaagaagcaccggctaactccgtgccagcagccgcggtaat

SEQ ID NO: 21 - DNA, 80 bp, Escherichia coli
gagtaaagttaatacctttgcgacgcgacgttacccgcagaagaagcaccggctaactccgtgccagcagccgcggtaat

SEQ ID NO: 22 - DNA, 25 bp, Artificial Sequence
  Artificial target
tgctcattgacgttacccgcagaag

SEQ ID NO: 23 - DNA, 25 bp, Artificial Sequence
  a mismatch
tgcttattgacgttacccgcagaag

SEQ ID NO: 24 - DNA, 25 bp, Artificial Sequence
  3 mismatch
tgctggatgacgttacccgcagaag

SEQ ID NO: 25 - DNA, 25 bp, Artificial Sequence
  5 mismatch
tgcgacgcgacgttacccgcagaag

SEQ ID NO: 26 - DNA, 25 bp, Artificial Sequence
  20 mismatch
tatacattccatagatctggatacc

(19) United States
(12) Patent Application Publication
(10) Pub. No.: US 2020/0217819 A1
(43) Pub. Date: Jul. 9, 2020

(54) MISMATCH BINDING OF TWO MOLECULES AND GENETIC SEQUENCES USING KINETIC MEASUREMENTS IN ISOTACHOPHORESIS

(71) Applicant: TECHNION RESEARCH & DEVELOPMENT FOUNDATION LIMITED, Haifa (IL)

(72) Inventors: Moran BERCOVICI, Haifa (IL); Rebecca KHALANDOVSKY, Nesher (IL)

(21) Appl. No.: 16/611,593

(22) PCT Filed: May 10, 2018
(86) PCT No.: PCT/IL2018/050513

§ 371 Date: Nov. 7, 2019

Related U.S. Application Data
(60) Provisional application No. 62/503,981, filed on May 10, 2017.

(51) Int. Cl. G01N 27/447; C12Q 1/6827

(52) U.S. Cl. CPC G01N 27/44756; C12Q 1/6827

(57) ABSTRACT

Provided is a system including: an antisense molecule; a DNA molecule, an RNA molecule, or a combination of DNA and RNA molecules; and an Isotachophoresis (ITP) system. Furthermore, the invention provides a method for sequence-specifically separating and/or identifying a nucleic acid molecule of interest by utilizing the system of the invention to separate and possibly label and/or detect/quantify a nucleic acid molecule of interest.

### FIELD OF INVENTION

This invention is directed to; inter alia, a system for sequence-specifically separating and/or identifying a nucleic acid molecule, comprising: a morpholino molecule; and an Isotachophoresis (ITP) separation system.

### BACKGROUND OF THE INVENTION

Isotachophoresis (“ITP”) is a variant of electrophoresis, characterized by the fact that separation is carried out in a discontinuous buffer system. Sample material to be separated is inserted between a “leading electrolyte” and a “terminating electrolyte” or mixed in any of these, the characteristic of these two buffers being that the leader has to have ions of net mobility higher than those of sample ions, while the terminator must have ions of net mobilities lower than those of sample ions. In such a system, sample components sort themselves according to decreasing mobilities from leader to terminator, in a complex pattern governed by the so-called Kohlrausch regulating function. The process has been described repeatedly, as for instance, Bier and Allgyer, Electrokinetic Separation Methods 443-69 (Elsevier/North-Holland 1979).

It is further characteristic of ITP that a steady state is eventually reached, where all components migrate at same velocity (hence the name) in sharply defined contiguous zones. Sample components can be separated in such a contiguous train of components by insertion of “spacers” with mobilities intermediary between those of the components one wishes to separate.

Isoelectric focusing (“IEF”), also sometimes called electrofocusing, is a powerful variant of electrophoresis. The principle of IEF is based on the fact that proteins and peptides, as well as most biomaterials, are amphoteric in nature, i.e., are positively charged in acid media and negatively charged in basic media. At a particular pH value, called the isoelectric point (PI), there is reversal of net charge polarity, the biomaterials acquiring zero net charge.

If such amphoteric materials are exposed to a d.c. current of proper polarity in a medium exhibiting a pH gradient, they will migrate, i.e., ‘focus’ toward the pH region of their PI, where they become virtually immobilized. Thus a stationary steady state is generated, where all components of the mixture have focused to their respective PIs.

The pH gradient is mostly generated ‘naturally’ i.e, through the electric current itself. Appropriate buffer systems have been developed for this purpose, containing amphoteric components which themselves focus to their respective PI values, thereby buffering the pH of the medium.

The two variants, IEF and ITP, differ in that IEF attains a stationary steady state whereas in ITP a migrating steady state is obtained. Thus, in IEF a finite length of migrating channel is always sufficient. In ITP, complete resolution may require longer migrating channels than is practical. In such case, the migrating components can be virtually immobilized by applying a counterflow, the rate of counterflow being matched to the rate of frontal migration of the sample ions. This is also known in the art.

IEF is most frequently carried out in polyacrylamide or agarose gels, where all fluid flow disturbances are minimized. ITP is most often carried out in capillaries. The sample is inserted at one end of the capillary, at the interface between leader and terminator, and the migration of separated components recorded by appropriate sensors at the other end of the capillary. Both such systems are used mainly for analytical or micro-preparative purposes.

ITP forms a sharp moving boundary between ions of like charge. The technique can be performed with anionic or cationic samples. The system quickly establishes a strong gradient in electric field at the ITP interface, due to the non-uniform conductivity profile. As per its name (from Greek, “isos” means “equal”, “takhos” means “speed”), TE and LE ions travel at the same, uniform velocity, as a result of the non-uniform electric field and conservation of current (this is the so-called “ITP condition”).

The ITP interface is self-sharpening: LE ions that diffuse into the TE zone experience a strong restoring flux and return to the leading zone (and vice versa for TE ions in the LE zone). Sample ions focus at this interface if their effective mobility in the TE zone is greater than those of the TE co-ions, and if their effective mobility in the LE zone is less than that of the LE co-ions. The self-sharpening and focusing properties of ITP contribute to the robustness of this technique and make ITP relatively insensitive to disturbances of the interface (e.g. due to pressure-driven flow or changes in geometry, such as contractions, expansions, and turns).

In peak mode ITP, sample ion concentrations are at all times significantly lower than LE and TE ion concentrations and therefore contribute negligibly to local conductivity. The distribution of sample ions is determined by the self-sharpening interface between neighboring zones (here the TE and LE) and the value of the sample effective mobility relative to these zones. Multiple sample ions focus within the same narrow ITP interface region as largely overlapping peaks.

Morpholinos are a neutral-backbone DNA analogue, with an affinity to DNA larger than that of DNA. morpholinos are used extensively in embryological mRNA knockdown studies.

### SUMMARY OF THE INVENTION

In one embodiment, the present invention provides a system comprising: a morpholino molecule; a DNA molecule, an RNA molecule, or a combination thereof; and an Isotachophoresis (ITP) system. In some embodiments, the morpholino molecule is labeled. In one embodiment, the label is a fluorescent label.

In a further embodiment, the present invention provides a method for separating and/or detecting a specific hybrid comprising a first molecule and a second molecule, comprising the steps of: contacting a mixture of molecules comprising said first molecule and said second molecule in a first solution and obtaining a hybrid; introducing the first solution into the ITP system, the ITP system comprises a second solution of high effective mobility leading electrolyte (LE) ions and a third solution of low effective mobility trailing electrolyte (TE); applying voltage between the LE and the TE, inducing a low electrical field in the leading electrolyte and a high electrical field in the terminating electrolyte; wherein only the hybrid focus at the sharp LE-TE interface in the ITP system, thereby separating and/or detecting/quantifying a specific hybrid.

In a further embodiment, the present invention provides a method for sequence-specifically separating and/or detecting a nucleic acid molecule, comprising the steps of: contacting a mixture of nucleic acid molecules with a morpholino having an anti-sense sequence of interest in a first solution and obtaining a nucleic acid molecule/morpholino hybrid; introducing the first solution into the ITP system, the ITP system comprises a second solution of high effective mobility leading electrolyte (LE) ions and a third solution of low effective mobility trailing electrolyte (TE); applying voltage between the LE and the TE, inducing a low electrical field in the leading electrolyte and a high electrical field in the terminating electrolyte; wherein the nucleic acid molecule/morpholino hybrid but not free morpholino focus at the sharp LE-TE interface in the ITP system, thereby sequence-specifically separating and/or detecting/quantifying a nucleic acid molecule.

In another embodiment, “sequence-specifically” is a contiguous nucleic acid sequence comprising at least 7 nucleotides. In another embodiment, “sequence-specifically” is a contiguous nucleic acid sequence comprising at least 10 nucleotides. In another embodiment, “sequence-specifically” is a contiguous nucleic acid sequence comprising or consisting 7 to 15,000 nucleotides. In another embodiment, “sequence-specifically” is a contiguous nucleic acid sequence comprising or consisting 10 to 10,000 nucleotides. In another embodiment, “sequence-specifically” is a contiguous nucleic acid sequence comprising or consisting 10 to 1,000 nucleotides. In another embodiment, “sequence-specifically” is a contiguous nucleic acid sequence comprising or consisting 10 to 500 nucleotides.

In a further embodiment, the present invention provides a method for sequence-specifically separating and/or detecting/quantifying a nucleic acid molecule, comprising the steps of: (a) contacting a mixture of nucleic acid molecules with an antisense molecule having a sequence of interest in a first solution and obtaining a nucleic acid molecule/labeled-antisense probe mismatch hybrid and a nucleic acid molecule/labeled-antisense probe match hybrid; (b) introducing the first solution into the ITP system, wherein the ITP system comprises a second solution of high effective mobility leading electrolyte (LE) ions and a third solution of low effective mobility trailing electrolyte (TE); (c) applying an electric field across the second solution and the third solution; (d) measuring a signal of an electromigrating nucleic acid molecule/labeled-antisense probe mismatch hybrid, an electromigrating nucleic acid molecule/labeled-antisense probe match hybrid or both in at least two time points or in at least two locations wherein at least one location is situated before the focusing zone along the electromigration direction; wherein a hybrid but not free labeled-antisense probe focus at the sharp LE-TE interface in the ITP system, wherein the TE has a higher mobility than the labeled-antisense probe and the TE has a lower mobility than the hybrid, thereby reducing false positive detection of a nucleic acid molecule/labeled-antisense probe mismatch hybrid.

In a further embodiment, the present invention provides a method for sequence-specifically detecting and/or separating a nucleic acid molecule, comprising the steps of: contacting a mixture of nucleic acid molecules with a morpholino having a sequence of interest in a first solution and obtaining hybrids; introducing the first solution comprising the hybrids resulting from step (a) into an ITP system, the ITP system comprises a second solution of high effective mobility leading electrolyte (LE) ions, a third solution of low effective mobility trailing electrolyte (TE), and a fourth solution of intermediate effective mobility electrolyte (IE); applying an electric field across the second solution and the third solution; wherein the hybrid but not free morpholino focus at the sharp LE-IE interface in the ITP system and the free morpholino but not the hybrids focus at the sharp IE-TE interface in the ITP system, wherein the IE has a higher mobility than the morpholino probe and the IE has a lower mobility than the hybrid, thereby sequence-specifically separating a nucleic acid molecule.

In a further embodiment, the present invention provides a kit comprising a morpholino having a sequence of interest, a solution for selectively hybridizing a nucleic acid molecule and the morpholino; a solution having high effective mobility leading electrolyte (LE); a solution having low effective mobility trailing electrolyte (TE); and instructions for separating and/or detecting a hybrid consisting the nucleic acid molecule and the morpholino.

### BRIEF DESCRIPTION OF THE DRAWINGS

FIG. 1. Graphs showing theoretical results demonstrating concentration of probe-DNA hybrids at the ITP interface as a function of time since the beginning of ITP, for different dissociation rates k_{off} and a fixed accumulation rate Φ˜10, demonstrating the three different regimes. For

$$\frac{{k}_{off}}{Φ} < 1 ,$$

concentration first increases, driven by accumulation, and then eventually decreases as dissociation takes over. The smaller the dissociation constant k_{off} the longer the delay of the inversion point at which the hybrid concentration begins to decrease. Left plot shows solutions for low dissociation rates with

$$\frac{{k}_{off}}{Φ} ≪ 1 ,$$

demonstrating that in such cases the inversion point takes place only after any relevant experiment will have ended (note log-log scale). Right plot shows solutions for

$$\frac{{k}_{off}}{Φ} < 1$$

in which the inversion point takes place within 10 seconds of beginning ITP. Inset focuses on the transient solutions at times shorter than 1 second yielded by high k_{off} values, including those in which

$$\frac{{k}_{off}}{Φ} \geq 1 ,$$

which leads to a continually-decreasing concentration as dissociation dominates from the start. In the calculation of

$$Φ ≡ \frac{( γ - 1 ) {u}_{I T P}}{δ} , \\ {μ}_{P D} = - 17 \cdot {10}^{- 9} \frac{{m}^{2}}{V \cdot s}$$

was obtained from the model proposed by Savard et al. using the parameters obtained by Ostromohov et al.³⁵, and

$${μ}_{T} = - 19 \cdot {10}^{- 9} \frac{{m}^{2}}{V \cdot s}$$

was calculated in SPRESSO, yielding

$$γ = \frac{{μ}_{P D}}{{u}_{T}} = 1.11 ;$$

μ_{ITP} ˜1 mm/s and δ˜10 μm were obtained from experimental observations of ITP run at a constant voltage of 1000 V on our experimental setup.

FIG. 2. Is a bar graph showing experimental results characterizing specificity of 25 nt morpholno probe using random and mismatch DNA targets at varying temperatures. The ITP-NFP assay was performed at 20° C., 40° C. 55°C. , 60° C., and 70° C. using 25 nt lissamine-labeled morpholino probes and 80 nt DNA targets containing a either (M) a 25 nt sequence perfectly matching the probe, (5) a 25 nt sequence matching the probe except for a 5 nt-long mismatch, or (R) two 4 nt-long stretches matching a single 4 nt region of the probe, to represent random binding. The results demonstrate a maximum one order of magnitude difference between the signals from the match and 5 nt mismatch targets at 55° C. At 40° C. and higher, the probe does not detect 4 nt long “random” binding, and at 20° C. the signal from this binding is 2 orders of magnitude less than that from the matching target. Probes and targets were each present at 10 nM, except in the control case (C) where neither was present. Lissamine fluorescence was excited using a metal halide light source. Each data point represents the area-averaged fluorescent intensity of the ITP plug, averaged over 3-6 frames, registered 18 mm from the TE reservoir. Each bar shows the mean intensity for all experiments performed at that condition, and uncertainty bars represent 95% confidence on this mean. Each data point obtained is shown, with “n=” indicating the total number of repeats per experimental condition. When no signal higher than the threshold value of mean +3 standard deviations of the background could be detected, the maximal value of this threshold was recorded as a “maximum undetected signal”; such “low signals” are represented by downward-facing triangles, and are averaged together with detected signals at the same experimental condition to obtain the mean intensity at that condition. The maximum value of the “maximum undetected signal” across all experiments defines the horizontal line indicating the limit of detection, showing that signals below that value cannot be reliably detected.

FIG. 3. Is a bar graph showing experimental results characterizing specificity of a 14 nt morpholno probe using random and mismatch DNA targets at different temperatures. The ITP-NFP assay was performed at 20° C., 40° C., and 55° C. using 14 nt lissamine-labeled morpholino probes and 80 nt DNA targets containing a either a 14 nt-long sequence perfectly matching the probe(Match), a 14 nt-long sequence matching the probe except for a 1-2 nt-long mismatch at the edge of the probe sequence (1-2mm, respectively), or two 4 nt-long stretches matching a single 4 nt region of the probe, to represent random binding (Rand). The results demonstrate that the 14 nt morpholino probe yields a ˜1.5 order of magnitude difference between the signals from the Match and 1-2 nt mismatch targets at 40° C. Probes and targets were each present at 10 nM, except in the control case (C) in which targets were absent. Lissamine fluorescence was excited using a metal halide light source. Each data point represents the area-averaged fluorescent intensity of the ITP plug, averaged over 3-6 frames, registered 18 mm from the TE reservoir. Each bar shows the mean intensity for all experiments performed at that condition, and uncertainty bars represent 95% confidence on this mean. Each data point obtained is shown, with “n=” indicating the total number of repeats per experimental condition. When no signal higher than the threshold value of mean +3 standard deviations of the background could be detected, the maximal value of this threshold was recorded as a “maximum undetected signal”; such experiments with “signal not detected” are represented by downward facing triangles, and are averaged together with detected signals at the same experimental condition to obtain the mean intensity at that condition. The maximum value of the “maximum undetected signal” across all experiments defines the horizontal line indicating the limit of detection, showing that signals below that value cannot be reliably detected.

FIG. 4. Is a bar graph showing experimental results demonstrating effect of probe concentration on signal. The ITP-NFP assay was performed at 20° C. using 25 nt lissamine-labeled morpholino probes and 80 nt DNA targets either containing a 25 nt region perfectly matching the probe (Match), or containing two 4 nt stretches complementary to a 4 nt region of the probe to simulate random binding (Rand). The results demonstrate a less than one order of magnitude decrease in signal as target concentration decreases from 10 nM to 1 nM for both the Match and Random targets, while the probe concentration is fixed at 10 nM. They also show an almost 2 orders of magnitude decrease in signal for the Match target as the concentration of the probe drops from 10 nM to 1 nM while the target concentration remains fixed at 1 nM; this is suspected to be due to the hybridization reaction not reaching equilibrium during the time allotted due to the lower concentration of probes. The control case (Ctrl) shows the signal with DNA absent. Lissamine fluorescence was excited using a metal halide light source. Each data point represents the area-averaged fluorescent intensity of the ITP plug, averaged over 5-6 frames, registered 18 mm from the TE reservoir. Each bar shows the mean intensity for all experiments performed at that condition, and uncertainty bars represent 95% confidence on this mean. Each data point obtained is shown, with “n=” indicating the total number of repeats per experimental condition. When no signal higher than the threshold value of mean +3 standard deviations of the background could be detected, the maximal value of this threshold was recorded as a “maximum undetected signal”; such “low signals” are represented by downward facing triangles. The mean of the maximum value of the “maximum undetected signal” and the minimum “detected signal” across all experiments defines the horizontal line indicating the limit of detection, showing that signals below that value cannot be reliably detected.

FIG. 5. Is a bar graph showing experimental results demonstrating specificity of 25 nt probe in the presence of a high concentration of non-target DNA. The ITP-NFP assay was performed at 20° C. using 25 nt lissamine-labeled morpholino probes and 80 nt DNA targets either containing a 25 nt region perfectly matching the probe (Match), or containing two 4 nt-long stretches complementary to a 4 nt region of the probe to simulate random binding (Random). The results demonstrate that 100 nM-10 μM concentrations of the Random target yield approximately identical signals, suggesting saturation of the probe; however, this signal is roughly 1 order of magnitude less than the signal from the Match at 10 nM, suggesting that steady state hybridization kinetics may not fully describe the situation. Probes were present at 10 nM and DNA was present at the concentrations indicated. Lissamine fluorescence was excited using a metal halide light source. Each data point represents the total fluorescent intensity of the ITP plug, averaged over 2-6 frames, registered 18 mm from the TE reservoir. Each bar shows the mean intensity for all experiments performed at that condition, and uncertainty bars represent 95% confidence on this mean. Each data point obtained is shown, with “n=” indicating the total number of repeats per experimental condition. When no signal higher than the threshold value of mean +3 standard deviations of the background could be detected, the maximal value of this threshold was recorded as a “maximum undetected signal”; such “low signals” are represented by downward facing triangles.

FIG. 6. Is an illustration of multi-detector device and system.

FIG. 7. Is a schematic illustration of the assay: a microfluidic channel connecting two reservoirs is initially filled with LE. The left reservoir is filled with a mixture of TE, nucleic acid (NA) sample and a high concentration of Morpholino probes. The high concentration of Morpholino probes results in rapid, but potentially non-specific, binding to NA sequences. The negatively charged hybrids electromigrate into the channel and focus at the ITP interface, while unbound, weakly charged Morpholino probes remain behind. All hybrids focus at the ITP interface. Hybrids with a high rate dissociate quickly, and unbound Morpholinos fall behind the interface. Additional mismatched hybrids progressively dissociate according to their off-rate, until for a sufficiently long time all non-specific hybrids are melted and the fluorescent signal is obtained from specific hybrids only.

FIG. 8. Are graphs showing experimental validation (symbols) of the accumulation-dissociation dynamics model (solid lines) using a 25 nucleotide-long probe and varying k_{off} by varying (A) the operating temperature, and (B) the number of bases matching the probe.

Monotonically increasing data was fitted; since at T_{ref}+15° C. the signal is no longer visible after 350 s (it likely drops significantly below the quantification threshold), there is insufficient data for a reliable fit at that condition. In all experiments, 100 nM fluorescently labeled Morpholino probe and 10 nM DNA were injected together with TE into the TE reservoir and applied a constant current of 1 μA. The dashed lines represent the quantitation threshold at each temperature, defined as 2 standard deviations above the mean of the negative control case (taken with probes but without DNA). The reference temperature T_{ref} was defined as the minimum temperature at which the 20/25 bp hybrid exhibits non-monotonic behavior.

FIG. 9. Are graphs showing Demonstration of the accumulation-dissociation assay's specificity and mismatch discrimination ability. Comparison of the signal (symbols) obtained from a match and (A) a 5-bp mismatch (20/25 bp) at 10-fold higher concentration, and (B) a minimally matching (4/25 bp) sequence at 1000-fold higher concentration. Despite a significantly higher concentration of mismatches, for sufficiently long times only the signal of the targets is visible over the noise. Solid lines correspond to a model prediction as described in FIG. 8, and horizontal dashed line represents the quantitation threshold as defined in FIG. 8.

FIG. 10. Are graphs showing analytical prediction of the assay's specificity. (A-B) Model-based simulations comparing the signal obtained from a mixture (solid black curves) of target sequences and an excess of mismatches, to that of the negative controls (mismatches only, solid gray curves) and that of the targets only (dashed curve). At early times, the signal of the mixture is indistinguishable from that of the negative control, whereas for sufficiently long times, the signal of the mixture follows that of the targets (linearly increasing; note log scale) as the signal of the negative control decays exponentially. Times at which the signal of the mixture exceeds that of the negative control by a factor of 3 are indicated by dashed vertical lines. An arbitrary value of the quantitation threshold (dash-dot line) was presented to illustrate that while operation at higher temperatures enables faster analysis time, absolute signals at those times may be undetectable. Higher detection sensitivity would thus enable specific detection at a shorter time. (C) Contour map showing time needed to achieve desired specificity. Each contour line corresponds to a single ratio of target to mismatch concentrations, from 1:1 to 1:10¹⁰. For each concentration ratio, the intersection of the contour line with the k_{off} value of the mismatch (y-axis) can be used to extract the minimal time at which the signal of the mixture of the target with that excess of mismatches could be distinguished from the negative control (mismatches only) with a 3:1 signal ratio. Horizontal lines represent estimated k_{off} for 1-bp and 3-bp mismatches at 60° C. and 65° C. (see S12 in the Supporting Information). For a conservative prediction, a match k_{off} value of 4·10⁻⁴ s⁻¹, was used as would be expected for a 25-bp DNA-DNA hybrid at 65° C. (see S12); at 60° C., this value decreases, resulting in earlier discrimination of the targets.

FIG. 11. Is a schematic of the NS12A microfluidic chip showing the 11 imaging locations (“stations”) along the channel, and the channel cross section (bottom right).

FIG. 12. A graph reproduced using values of γ=1.3, 1.6, 1.8, 2.0, 2.3 on the same plot. The resulting curves are nearly identical, showing insensitivity of the current results to the value of γ on this range.

FIG. 13. Are graphs showing experimental characterization (symbols) of the onset of rehybridization at the ITP interface at varying DNA concentrations and at (A) 1 μA and (B) 2 μA applied current. Rehybridization and the halt in signal decay begin after the amount of DNA at the interface reaches N_{bs,crit}^{ITP}. For each condition, dashed curves model total DNA at the interface, and vertical lines show the times t_{crit}, at which N_{bs,crit}^{ITP} is reached. For 1 nM at 2 μA, N_{bs,crit}^{ITP} is not reached during the experimental time. Horizontal dashed line represents the quantitation threshold.

FIGS. 14-18. Are DNA sequences of tables 1-4 and list of A to E, accordingly. Shading shows bases present in 14 nt probe; underlining shows bases present in 25 nt probe.

### DETAILED DESCRIPTION OF THE INVENTION

In one embodiment, the present invention provides a system comprising: (A) a probe or a morpholino molecule; (B) a nucleic acid molecule; and (C) an Isotachophoresis (ITP) system. In another embodiment, an ITP system comprises: a first zone comprising a solution of high effective mobility leading electrolyte (LE) ions; a second zone comprising a solution of low effective mobility trailing electrolyte (TE); and an anode and a cathode.

In one embodiment, provided a system comprising a microfluidic channel connecting two reservoirs. In one embodiment, the microfluidic channel comprises LE. In one embodiment, a reservoir of the two reservoirs comprises a mixture of TE, a nucleic acid (NA), and a probe or a Morpholino probe. In one embodiment, a reservoir of the two reservoirs comprises a mixture of TE, a nucleic acid (NA), a hybrid and a probe or a Morpholino probe. In one embodiment, a probe or a Morpholino probe is in a concentration of above 10 nM.

In one embodiment, a high concentration of a probe or a Morpholino probe results in rapid, but potentially non-specific, binding to a NA sequence. In one embodiment, binding of a probe or a morpholino and a NA results in the formation of a hybrid. In one embodiment, a hybrid is negatively charged.

In one embodiment, a negatively charged hybrid electromigrates into the channel and focuses at the ITP interface, while unbound, weakly charged probe or a Morpholino probe remains behind.

In one embodiment, a hybrid with a high rate of mismatch between the sense (the analyte) and the antisense (probe and/or Morpholino) dissociates quickly, and the unbound a probe or a Morpholino probe falls behind the interface. In one embodiment, an unperfect hybrid is a hybrid comprising a high rate of mismatch. In one embodiment, an unperfect hybrid is a hybrid comprising at least one mismatch between the hybrid and the NA. In one embodiment, an unperfect hybrid is a hybrid comprising at least two mismatches between the hybrid and the NA. In one embodiment, an unperfect hybrid is a hybrid comprising at least three mismatches between the hybrid and the NA. In one embodiment, an unperfect hybrid is a hybrid comprising at least five mismatches between the hybrid and the NA. In one embodiment, an unperfect hybrid is a hybrid comprising at least seven mismatches between the hybrid and the NA. In one embodiment, an unperfect hybrid is a hybrid comprising at least ten mismatches between the hybrid and the NA. In one embodiment, an unperfect hybrid is a hybrid comprising at least 1% and no more than 50% mismatches between the hybrid and the NA. In one embodiment, an unperfect hybrid is a hybrid comprising at least 1% and no more than 20% mismatches between the hybrid and the NA. In one embodiment, an unperfect hybrid is a hybrid comprising at least 1% and no more than 10% mismatches between the hybrid and the NA. In one embodiment, an unperfect hybrid is a hybrid comprising at least 3% and no more than 15% mismatches between the hybrid and the NA. In one embodiment, the percentages recited apply to the percent on nucleotides with the NA that mismatched with a probe or a morpholino within a hybrid.

In one embodiment, a hybrid with a high k_{off} rate dissociates rapidly or quickly (FIG. 7). In one embodiment, an unbound probe or unbound Morpholino fall behind the interface (t₃) (FIG. 7). In one embodiment, a hybrid progressively dissociates according to its off-rate, until time t₄ when a hybrid is melted (FIG. 7). In one embodiment, a non-specific hybrid is melted and/or all non-specific hybrids are melted and the fluorescent signal is obtained from a specific hybrid/s only (FIG. 7). In one embodiment, the higher: rate, number or percentage of mismatch causes a hybrid progressively to dissociate according to its off-rate.

In one embodiment, according to the methods as described herein a sample to be tested is injected into the TE reservoir together with a high enough concentration of a probe or a morpholino probe which ensures immediate hybridization to all target NA sequences present in the sample. In one embodiment, the mobility of the terminating ions pμ_{T} is chosen to be higher than that of the free a probe or Morpholino, but less than that of the probe or Morpholino-NA hybrid μ_{PD} such that when an electric field is established or applied along the channel and ITP focusing is initiated, only a probe or a Morpholino-NA hybrid focuses at the TE/LE interface while a free (unbound to NA) Morpholino remains unfocused. In one embodiment, once probe-NA hybrid or morpholino-NA hybrid is brought into the channel by the electric field, it leaves the equilibrium state established in the TE reservoir: since no free probes are available in the channel or focused zone, no new association can take place, while a hybrid is subject to dissociation governed by its off-rate. In one embodiment, a probe or a morpholino that dissociate from a focused hybrid does not have a sufficiently high mobility to remain focused and is left behind, at the same time, an additional hybrid continues to leave the reservoir and undergo dissociation on its way to the ITP interface.

In another embodiment, the nucleic acid molecule is DNA, RNA, miRNA, mRNA, tRNA, or rRNA. In another embodiment, the leading electrolyte (LE) buffer is chosen such that its anions have higher effective electrophoretic mobility than the anions of the trailing electrolyte (TE) buffer (Effective mobility describes the observable drift velocity of an ion and takes into account the ionization state of the ion, as described in detail by Persat et al.). In another embodiment, sample ions of intermediate effective mobility race ahead of TE ions but cannot overtake LE ions, and so they focus at the LE-TE interface (hereinafter called the “ITP interface”). In another embodiment, the LE and TE buffers are chosen such that nucleic acid molecule of interest (to be detected or separated) have a higher mobility than the TE, but cannot over-speed the LE. In another embodiment, the TE and LE buffers form regions of respectively low and high conductivity, which establish a steep electric field gradient at the ITP interface. In another embodiment, the LE buffer (or LE) has a high ionic strength. In another embodiment, Mg²⁺ ions are used as a counter ion to promote rapid hybridization. In another embodiment, TE buffer (or TE) comprises MES (2-(N-morpholino)ethanesulfonic acid). In another embodiment, LE comprises hydrochloric acid. In another embodiment, LE comprises 70 to 150 mM HCl and 150 to 280 mM Bistris (2,2-Bis(hydroxymethyl)-2,2′, 2″ -nitrilotriethanol).

In one embodiment, a system, a method or a kit as described herein further comprises a IE ions and/or IE buffer. In one embodiment, IE is intermediate effective mobility electrolyte (IE). In one embodiment, IE has a higher mobility than the morpholino probe. In one embodiment, IE has a lower mobility than the hybrid. In one embodiment, IE has a higher mobility than the morpholino probe and a lower mobility than the hybrid. In one embodiment, IE is mixed with a first solution, a second solution, a third solution, or any combination thereof, as described herein. In one embodiment, IE comprises MES. In one embodiment, IE comprises MES and TE comprises tricine. In one embodiment, IE comprises MES and TE glycine.

In another embodiment, TE has a higher mobility than the unbound morpholino probe. In another embodiment, TE has a lower mobility than the nucleic acid molecule/morpholino hybrid. In another embodiment, LE has a higher mobility than the nucleic acid molecule/morpholino hybrid. In another embodiment, LE has a higher mobility than the nucleic acid molecule/morpholino hybrid, the nucleic acid molecule/morpholino hybrid has a higher mobility than TE, and TE has a higher mobility than the unbound morpholino molecule.

In another embodiment, LE comprises hydrochloric acid. In another embodiment, LE comprises 70 to 100 mM HCl. In another embodiment, LE comprises hydrochloric acid. In another embodiment, LE comprises 100 to 150 mM HCl. In another embodiment, LE comprises hydrochloric acid. In another embodiment, LE comprises 120 to 150 mM HCl. In another embodiment, LE comprises hydrochloric acid. In another embodiment, LE comprises 150 to 200 mM Bistris. In another embodiment, LE comprises 200 to 250 mM Bistris. In another embodiment, LE comprises 150 to 200 mM Bistris. 220 to 280 mM Bistris.

In another embodiment, in peak mode ITP with sample mixed in the TE reservoir, the amount of accumulated sample at the ITP interface, N_{α}, is determined by the ratio of the electrophoretic mobility of the analyte, μ_{α}, and of the TE, μ_{TE},

$${N}_{a} ∼ \frac{{μ}_{a}}{{μ}_{TE}} - 1.$$

In another embodiment, tricine with bistris is utilized as the TE buffer yielding a trailing ion mobility of 5.68·10⁻⁹ [m² V⁻¹s⁻¹]. In another embodiment, the mobility of TE is lower than the mobility of the nucleic acid molecule of the invention. In another embodiment, morpholino molecule has a neutral or slight positive charge. In another embodiment, selective focusing of morpholino-nucleic-acid-molecule complexes, but not free morpholino requires specific choice of an ITP system comprised of a TE buffer with sufficient mobility to over-speed the free morpholino probes but not the morpholino-nucleic-acid-molecule complexes (μ_{PNA}<μ_{TE}<μ_{complex}).

In another embodiment, according to the system, kits and methods of the invention no signal for the free morpholino molecule is obtained while maintaining a significant signal when nucleic acid molecule complexes, are present.

In another embodiment, ITP includes a microchannel connected to two reservoirs and is initially filled with LE solution. In another embodiment, a sample comprising a nucleic acid molecule to be detected is mixed in the trailing electrolyte (TE) reservoir. In another embodiment, a sample comprising a nucleic acid molecule to be detected is mixed in the leading electrolyte (LE) reservoir. In another embodiment, an electric field induces the electromigration of all ions in the channel.

In another embodiment, morpholino is an artificial DNA analogue in which the natural negatively charged deoxyribose phosphate backbone has been replaced by a synthetic neutral pseudo peptide backbone. In another embodiment, the four natural nucleobases are retained on the backbone at equal spacing to the DNA bases. In another embodiment, morpholino is substituted with another molecule which hybridizes according to base pairing (biologically stable molecule capable of sequence specific binding to DNA and RNA) with a nucleic acid molecule and renders the hybridized nucleic acid molecule weakly charged or uncharged. In another embodiment, the present invention takes advantage of morpholino's hybridization properties and specificity, and utilizes morpholino as a highly selective biosensor for nucleic acid sequence detection.

In another embodiment, morpholino comprise the formula:

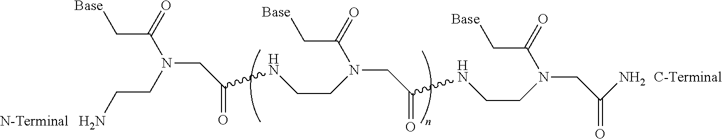

In another embodiment, a morpholino molecule as described herein comprises at least one lysine residue. In another embodiment, a morpholino molecule as described herein is further modified with at least one lysine residue. In another embodiment the concentration of morpholino is from 1 nM to 120 μM. In another embodiment the concentration of morpholino is from 2 nM to 500 nM. In another embodiment the concentration of morpholino is from 500 nM to 2 μM. In another embodiment the concentration of morpholino is from 2 μM to 30 μM.

In one embodiment, a probe or a Morpholino probe is in a concentration of 1 nM to 500 μM. In one embodiment, a probe or a Morpholino probe is in a concentration of 10 nM to 100 μM. In one embodiment, a probe or a Morpholino probe is in a concentration of 50 nM to 10 μM. In one embodiment, a probe or a Morpholino probe is in a concentration of 10 nM to 1 μM. In one embodiment, a probe or a Morpholino probe is in a concentration of 5 nM to 500 nM.

In one embodiment, a probe or a Morpholino probe is in a concentration of at least 1 nM. In one embodiment, a probe or a Morpholino probe is in a concentration of at least 10 nM. In one embodiment, a probe or a Morpholino probe is in a concentration of at least 50 nM. In one embodiment, a probe or a Morpholino probe is in a concentration of less than 500 μM. In one embodiment, a probe or a Morpholino probe is in a concentration of less than 100 μM. In one embodiment, a probe or a Morpholino probe is in a concentration of less than 50 μM. In one embodiment, a probe or a Morpholino probe is in a concentration of less than 10 μM.

In another embodiment, n equals 7 to 15,000. In another embodiment, n equals 10 to 10,000. In another embodiment, n equals 20 to 35,000. In another embodiment, n equals 10 to 1,000.

In another embodiment, morpholino comprise primary amine at the N-terminal. In another embodiment, morpholino further comprises a label. In another embodiment, the N-terminal primary amine of the morpholino is labeled. In another embodiment, morpholino is used as a probe.

In another embodiment, labeled morpholino is a morpholino molecule labeled with a positively charged molecule. In another embodiment, labeled morpholino is a morpholino molecule labeled with a cationic marker. In another embodiment, labeled morpholino comprises a fluorescent label. In another embodiment, labeled morpholino comprises a radioactive label. In another embodiment, labeled morpholino comprises a chemiluminescent label. In another embodiment, labeled morpholino comprises a colorimetric label. In another embodiment, morpholino serves as a probe, as an electrical charge quencher, as a specific antisense molecule for the identification and/or separation of a nucleic acid molecule having a specific sequence of interest (the target nucleic acid molecule).

In one embodiment, a system and/or a method as described herein includes a sample comprising a target nucleic acid molecule also referred to as a “target sequence” and/or “analyte”. In one embodiment, a system as described herein comprises a TE reservoir (comprising the TE solution) and a LE reservoir (comprising the LE solution). In one embodiment, the LE solution, the TE solution or both comprise or mixed with at least one target sequence. In one embodiment, a system and/or a method as described herein include a morpholino probe. In one embodiment, a system and/or a method as described herein include a non-focusing morpholino probe.

In one embodiment, a system as described herein comprises counterflow means such as a pressure device or a pump. In one embodiment, a system as described herein comprises a heating means. In one embodiment, heating means is used for providing stringent hybridization condition as further described herein. In one embodiment, a system as described herein comprises a thermometer. In one embodiment, a system as described herein comprises a pH meter.

In one embodiment, utilization of morpholino probes, in a method and a system as described herein, is unexpectedly efficient compared to other antisense probes such as but not limited to PNA.

In one embodiment, the terms “antisense”, “antisense molecule” or “probe” are used interchangeably. In one embodiment, “antisense”, “antisense molecule” or “probe” are labelled.

In one embodiment, “antisense”, “antisense molecule” or “probe” is an oligomer or a molecule which binds specific sequence of a nucleic acid molecule (sense). In one embodiment, “antisense”, “antisense molecule” or “probe” mimics a nucleic acid molecule—antisense, having sequence specificity to a sense-target, nucleic acid molecule. In one embodiment, “antisense”, “antisense molecule” or “probe” is an oligomer or a molecule complementary to a nucleic acid molecule having a sequence of interest. In one embodiment, a nucleic acid molecule having a sequence of interest is a target nucleic acid molecule. In one embodiment, “antisense”, “antisense molecule” or “probe” of the invention act by “steric blocking”, binding to a target sequence (“target of interest”) within a nucleic acid molecule. In one embodiment, “antisense”, “antisense molecule” or “probe” is radioactive labeled or labeled with a dye. In one embodiment, “antisense”, “antisense molecule” or “probe” comprises a PNA, a morpholino, a DNA, a RNA or any combination thereof

In one embodiment, the hybridization step for forming a hybrid as described herein requires specific hybridization conditions that ensure specific hybridization between the target and the antisense. In one embodiment, the hybridization step for forming a match-hybrid as described herein requires specific hybridization conditions that ensure specific hybridization between the target and the antisense. In one embodiment, the hybridization step for forming hybrid as described herein reduces the occurrence or formation of mismatch hybrids.

In one embodiment, hybridization is performed in a first solution. In one embodiment, the first solution is a buffer. In one embodiment, hybridization is performed in a first solution. In one embodiment, the first solution is LE, TE or a mixture thereof. In one embodiment, the first solution is adapted to control the efficiency of the hybridization step. In one embodiment, the first solution, second solution, third solution, or any combination thereof is/are maintained at a temperature just below the melting point (Tm) of the hybrid. In one embodiment, the first solution, second solution, third solution, or any combination thereof is/are maintained at a temperature which is 0.1 to 8° C. below the melting point (Tm) of the hybrid. In one embodiment, the first solution, second solution, third solution, or any combination thereof is/are maintained at a temperature which is 0.1 to 5° C. below the melting point (Tm) of the hybrid. In one embodiment, the first solution, second solution, third solution, or any combination thereof is/are maintained at a temperature which is 0.1 to 2.5° C. below the melting point (Tm) of the hybrid. In one embodiment, the hybrid is a match hybrid.

In one embodiment, the first solution, second solution, third solution, or any combination thereof is/are maintained at a pH value which ensures stringent hybridization condition that reduces the formation of mismatch hybrids. In one embodiment, the first solution, second solution, third solution, or any combination thereof includes a cation (such as a monovalent cation) at a concentration which ensures stringent hybridization condition that reduces the formation of mismatch hybrids. In one embodiment, the first solution, second solution, third solution, or any combination thereof includes an organic solvent at a concentration which ensures stringent hybridization condition that reduces the formation of mismatch hybrids. In one embodiment, the first solution, second solution, third solution, or any combination thereof is/are maintained at a temperature of 37⁺/−2.5° C. during the hybridization step.

In one embodiment, specific hybridization is characterized by high stringency, such as but not limited to: high hybridization temperature and low salt in hybridization buffers. In one embodiment, high stringency permits only hybridization between the target nucleic acid sequence and the probe/antisense. In one embodiment, high stringency only permits hybridization between nucleic acid molecules having a sequence that is at least 80% identical to the target nucleic acid sequence and the probe/antisense. In one embodiment, high stringency only permits hybridization between nucleic acid molecules having a sequence that is at least 85% identical to the target nucleic acid sequence and the probe/antisense.

In one embodiment, high stringency only permits hybridization between nucleic acid molecules having a sequence that is at least 90% identical to the target nucleic acid sequence and the probe/antisense. In one embodiment, high stringency only permits hybridization between nucleic acid molecules having a sequence that is at least 95% identical to the target nucleic acid sequence and the probe/antisense. In one embodiment, high stringency only permits hybridization between nucleic acid molecules having a sequence that is at least 97% identical to the target nucleic acid sequence and the probe/antisense. In one embodiment, high stringency conditions can be readily determined by one of average skill in the art, based on the sequence of the target to be detected (the amount/ratio of A/T and G/C bases and the length of the sequence).

In one embodiment, the first solution, second solution, third solution, or any combination thereof is/are maintained at a temperature of 37⁺/−3.5° C. throughout electro-migration within the ITP. In one embodiment, the first solution, second solution, third solution, or any combination thereof is/are maintained at a temperature of 37⁺/−2.5° C. throughout electro-migration within the ITP. In one embodiment, the first solution, second solution, third solution, or any combination thereof is/are maintained at a temperature of 37⁺/−2.0° C. throughout electro-migration within the ITP. In one embodiment, the first solution, second solution, third solution, or any combination thereof is/are maintained at a temperature of 37⁺/−1.5° C. throughout electro-migration within the ITP. In one embodiment, the first solution, second solution, third solution, or any combination thereof is/are maintained at a temperature of 37⁺/−1° C. throughout electro-migration within the ITP. In one embodiment, the first solution, second solution, third solution, or any combination thereof is/are maintained at a temperature of 37⁺/−0.5° C. throughout electro-migration within the ITP.

In one embodiment, the first solution, second solution, third solution, or any combination thereof includes a ssDNA which ensures stringent hybridization condition that reduces the formation of mismatch hybrids. In one embodiment, the first solution, second solution, third solution, or any combination thereof includes a tRNA which ensures stringent hybridization condition that reduces the formation of mismatch hybrids. In one embodiment, the first solution, second solution, third solution, or any combination thereof includes a polyA which ensures stringent hybridization condition that reduces the formation of mismatch hybrids. In one embodiment, the first solution, second solution, third solution, or any combination thereof includes a Denhardts solution which ensures stringent hybridization condition that reduces the formation of mismatch hybrids.

In one embodiment, probes (such as PNA or morpholino) are used in high concentration to guarantee an extremely fast reaction, such that all target sequences/analyte are rapidly bound to probes. In one embodiment, a probe comprises an antisense molecule. In one embodiment, an antisense molecule is not labeled. In one embodiment, a probe comprises an antisense molecule. In one embodiment, an antisense molecule (or antisense) is a labeled-antisense molecule. In one embodiment, high concentration of a probe results in binding of non-target sequences which results in false detection—a positive error. In one embodiment, the requirement of a high concentration of probe to obtain a signal renders ITP based probing assays, inoperative due to non-specific detection (false positive). In one embodiment, upon initiation of the ITP process, by providing an electrical current, free probe remain in the reservoir, and probe-DNA (both matches and mismatches) electromigrate into the channel and focus under ITP. In one embodiment, upon initiation of the ITP process by providing an electrical current, hybrids entering the channel begin to dissociate, with mismatches dissociating at much faster rate than matches (as determined by their off-rates). In one embodiment, probes that dissociated from mismatches do not have the required mobility to continue into the ITP interface and thus do not contribute to the signal. In one embodiment, probes that dissociated after reaching the ITP interface do not have the required mobility to remain focused. In one embodiment, probes that dissociated after reaching the ITP interface fall behind.

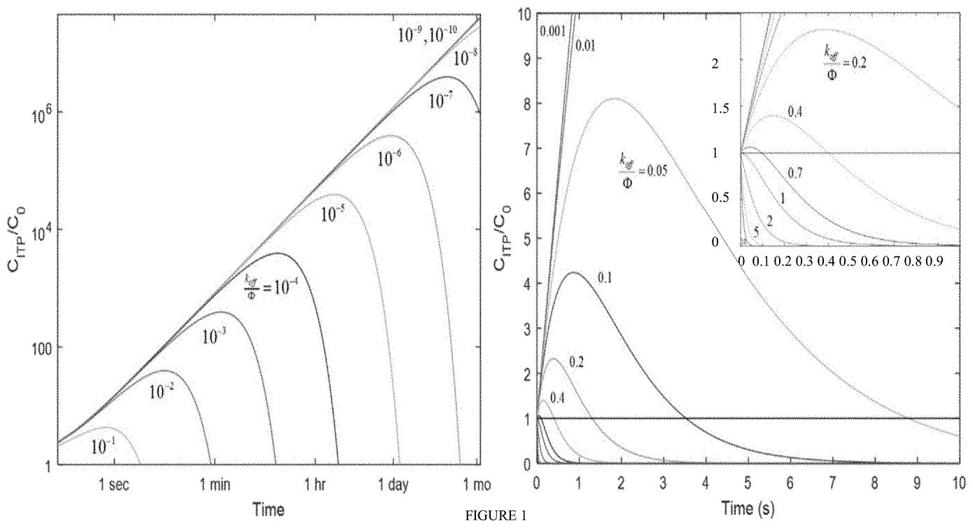

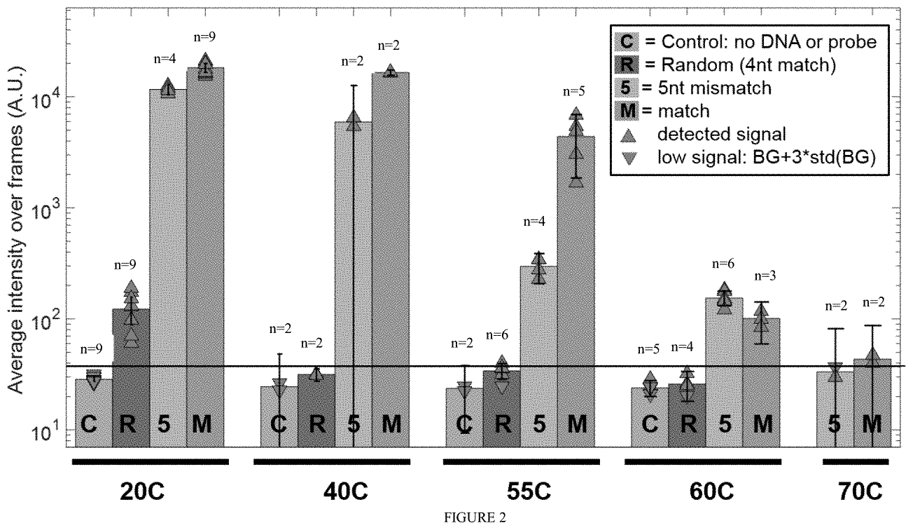

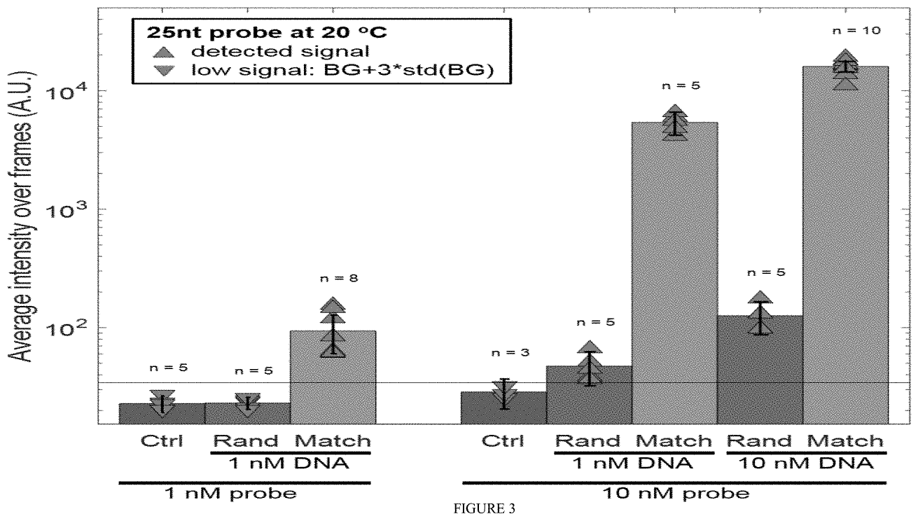

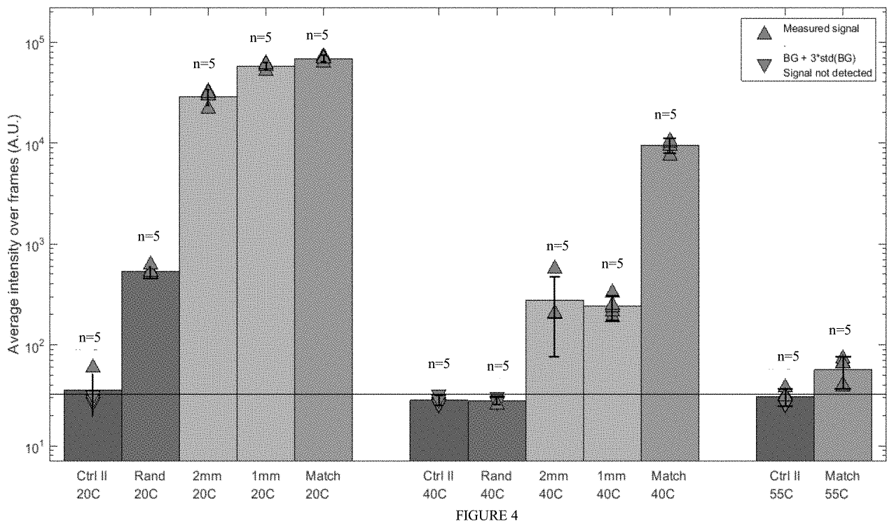

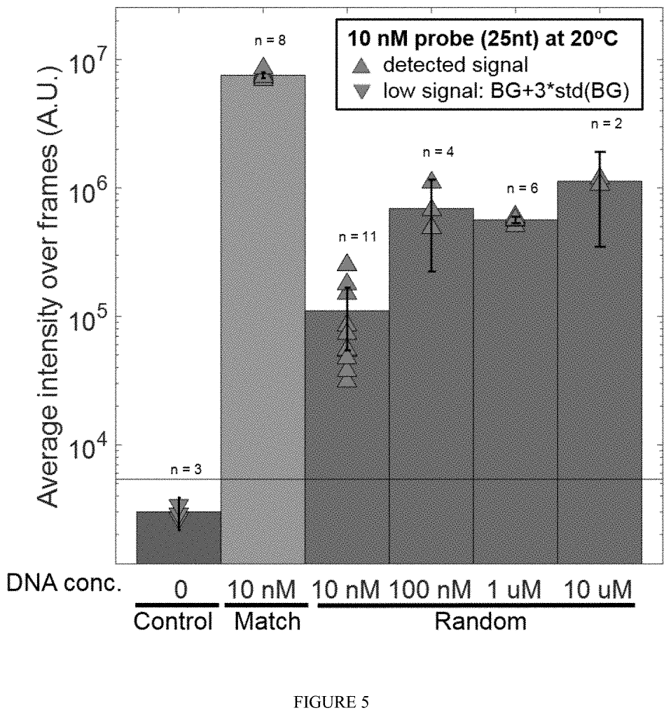

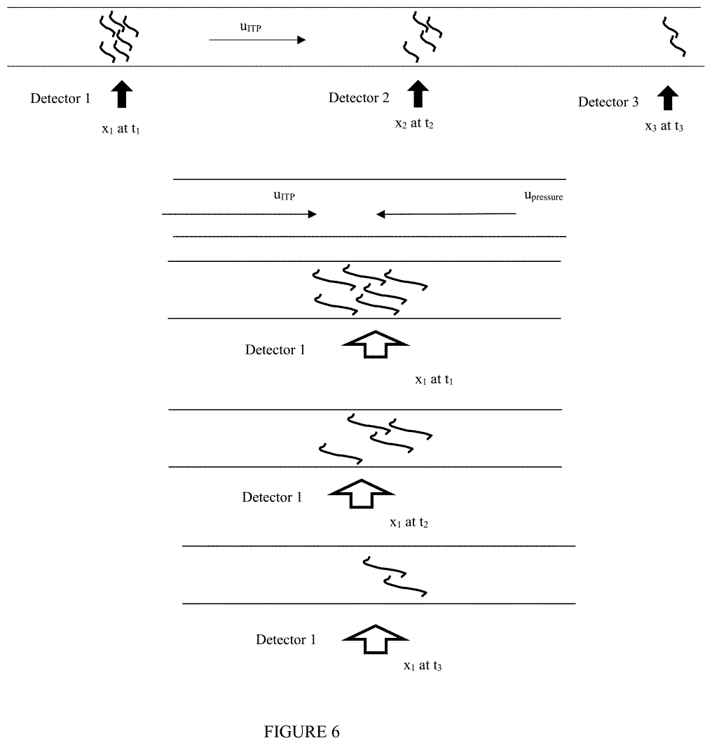

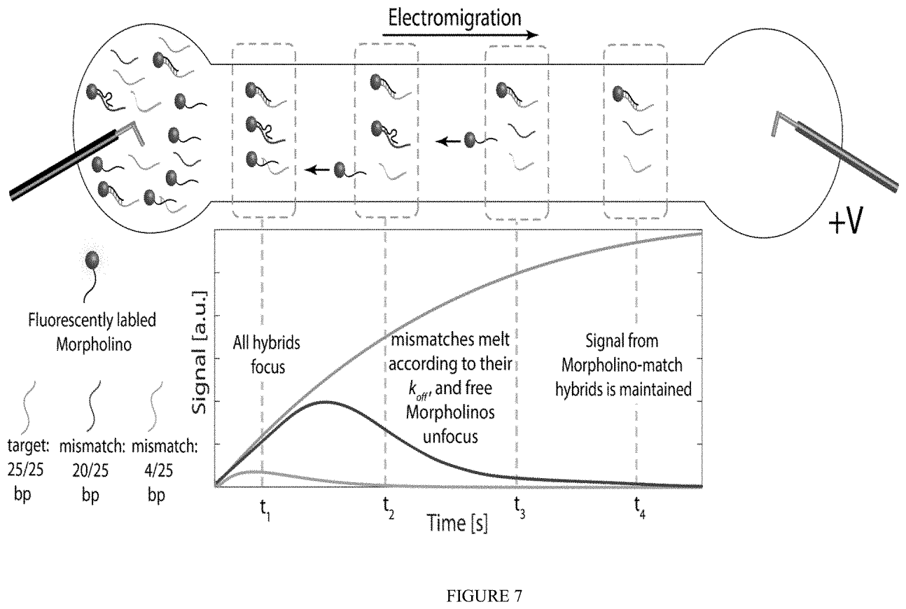

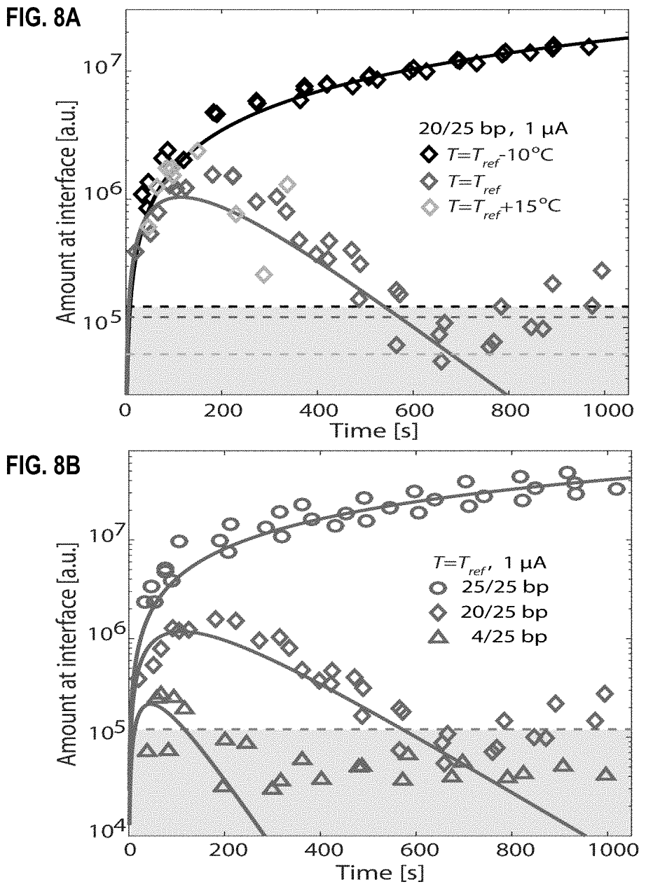

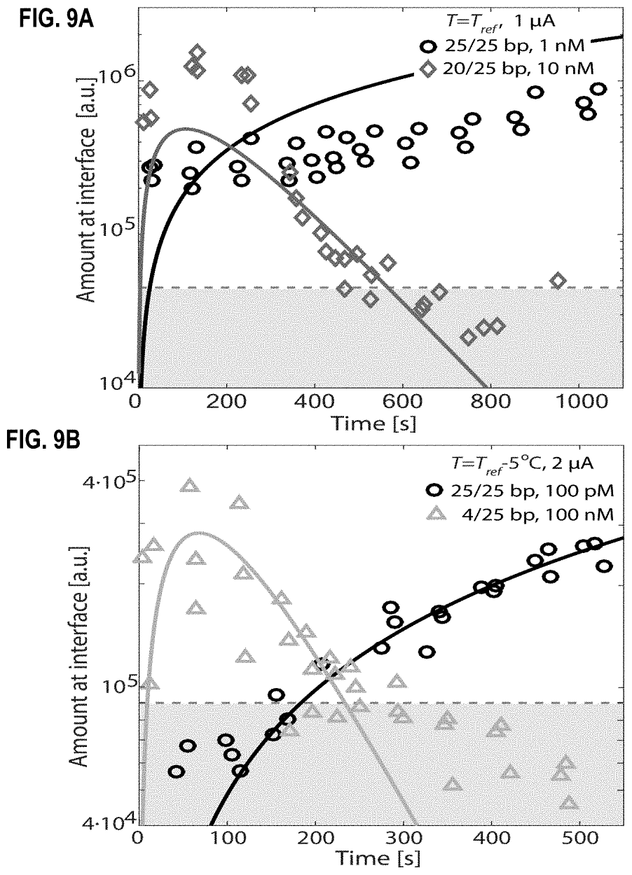

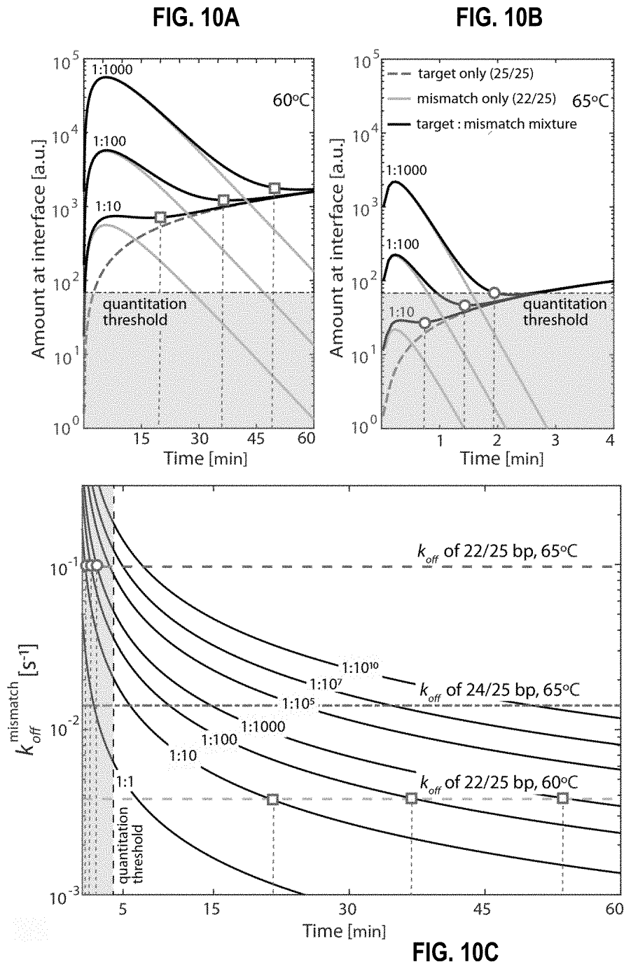

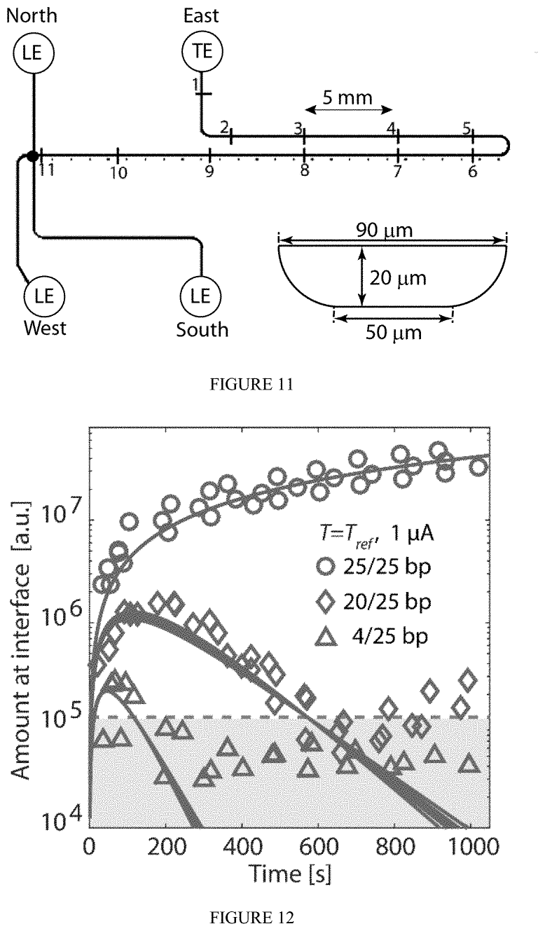

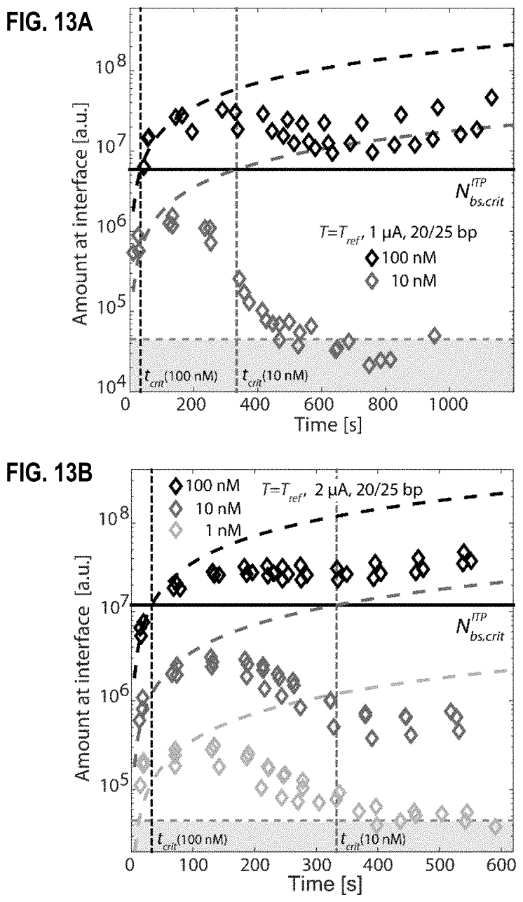

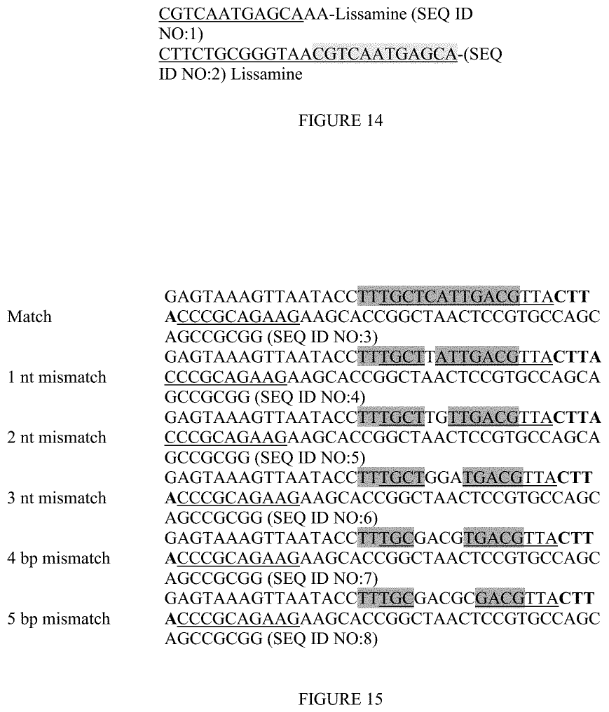

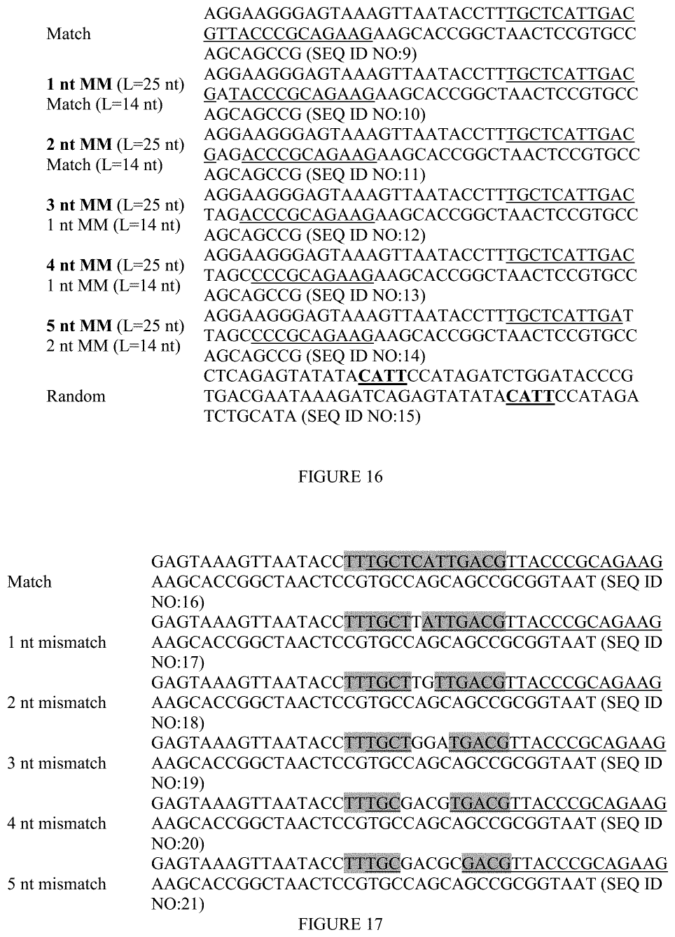

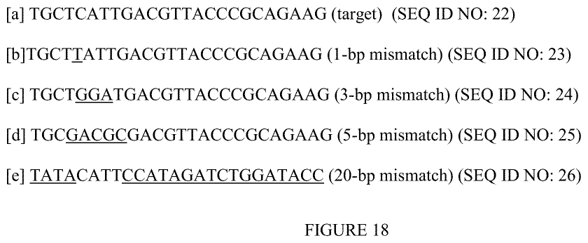

The signal (corresponding to the concentration of probes that remain associated) is given by:

$${c}_{P D}^{I T P} = {c}_{0} [ ( 1 - \frac{Φ}{{k}_{off}} \frac{γ}{( γ - 1 )} ) {e}^{- {k}_{off} t} + \frac{Φ}{{k}_{off}} \frac{γ}{( γ - 1 )} {e}^{- \frac{{k}_{off}}{γ} t} ] ,$$

where γ≡μ_{PD}/μ_{T} is the ratio of hybrid mobility to TE mobility, and Φ is a measure of the accumulation rate, given by Φ=(γ−1)u_{ITP}/δ, where u_{ITP} is the ITP velocity, and δ is the width of the interface. This solution has three different regimes, depending on the relation between the dissociation rate k_{off} and the accumulation rate ϕ For high dissociation rates k_{off}>ϕ the concentration of the hybrids at the ITP interface only decreases with time, whereas for lower dissociation rates k_{off}<ϕ the concentration increases to a maximum at tn. and then decreases, and for extremely low dissociation rates k_{off}>ϕ the concentration only increases (see FIG. 1).

In one embodiment, in the adjusted TE zone, between the reservoir (X=0) and the ITP interface (X=X^{ITP}) hybrids move with speed U_{PD-μPD}E^{TE} while dissociating at a rate k_{off}. In one embodiment, the interface migrates at velocity U_{ITP=μT}E^{TE}. Thus, hybrids travel for a duration of x^{ITP}/u_{PD}=(μ_{T}/μ_{PD})t before reaching the ITP interface, and enter it at a concentration of

$${c}_{PD} {\vert}_{{x}^{ITP}} ≡ {c}_{P D}^{ITP} ( t ) = η {c}_{P D}^{w} {e}^{- {k}_{off} t / γ} ,$$

where γ≡μ_{PD}/μ_{T}>1, c_{PD}^{w} is the hybrid concentration in the TE reservoir (assumed constant), t is the time since the application of voltage, and η=(μ_{PD}^{w}μ_{T}σ^{LE})/(μ_{PD}μ_{L}^{LE} σ^{w}) accounts for adaptation of the sample concentration from the reservoir to the adjusted TE zone. E, O and μ denote the electric field, solution conductivity, and species electrophoretic mobility, respectively, with subscripts referring to species (D—free NA, PD—probe-NA hybrid, T—terminating and L—leading ion), and superscripts referring to zones (w—TE reservoir, ITP—interface zone, LE—leading electrolyte zone, TE or none—adjusted terminating electrolyte zone). The rate of hybrid accumulation at the interface is given by dN_{PD}^{ITP}/dt=(γ−1)u_{ITP}A·c_{PD}(x^{ITP} (t)), where A is the cross-sectional area of the channel. Incorporating dissociation, the change in number of hybrids (in moles) at the interface is:

$$\frac{d {N}_{P D}^{ITP}}{d t} = ( γ - 1 ) {u}_{ITP} A η {c}_{P D}^{W} {e}^{- {k}_{off} t / γ} - {k}_{off} {N}_{P D}^{ITP} .$$

Solving this equation under the initial condition N(t=0)=0 in the channel yields the time-dependent number of hybrids (in moles) at the ITP interface:

$${N}_{P D}^{ITP} ( t ) = γ {u}_{ITP} A η {k}_{off}^{- 1} ( {e}^{- {k}_{off} t / γ} - {e}^{- {k}_{off} t} ) {c}_{P D}^{W} .$$

The second exponent corresponds to the dissociation of hybrids, whereas the first exponent corresponds to their accumulation. This accumulation rate depends on the concentration of hybrids arriving to the interface; as the interface moves further away from the TE reservoir, hybrids have a longer time to dissociate and thus arrive at a lower concentration. The maximum amount (or maximum concentration, assuming a uniform channel cross section and constant ITP interface width) is achieved when dN_{PD}^{ITP}/dt=0 at time

$${t}_{max} = \frac{γ ln γ}{( γ - 1 ) {k}_{off}} .$$

Prior to this time, accumulation dominates, and after it, dissociation dominates. It can be shown that dissociation is negligible (k_{off}→0) for k_{off}t Inγ/(γ−1) and in this case yields the well-known linear solution N_{PD}^{ITP} (t)=(γ−1)u_{ITP}Aηc_{PD}^{w}t. Thus, there is a time at which hybrids with higher k_{off} values (e.g. mismatches) will already yield a non-monotonic concentration characterized by their off-rate, while hybrids with sufficiently low k_{off} values (e.g. targets) will still yield a continuously increasing concentration at the ITP interface.

In one embodiment, the present invention provides a method and a system which reduces false positive detection of a mismatch in a hybrid or a heterohybrid comprising an analyte/target sequence bound to an antisense molecule. In one embodiment, a method which reduces false positive detection includes measuring a signal of a hybrid in at least two time points. In one embodiment, a method which reduces false positive detection includes measuring a signal of a hybrid in at least two locations wherein at least one location is situated before the focusing zone (in the electromigration direction).

In one embodiment, the phrase “electromigration direction” is interchangeable with the phrase “electromigration path”. In one embodiment, “electromigration direction” or “electromigration path” is from the anode to the cathode. In one embodiment, “electromigration direction” or “electromigration path” is from the cathode to the anode. In one embodiment, “electromigration direction” is from the TE reservoir to the LE reservoir. In one embodiment, “electromigration direction” or “electromigration path” is from the LE reservoir to the TE reservoir. In one embodiment, “electromigration direction” or “electromigration path” is from the TE solution to the LE solution. In one embodiment, “electromigration direction” or “electromigration path” is from the LE solution to the TE solution. In one embodiment, “electromigration direction” or “electromigration path” is from the anode to the focusing zone. In one embodiment, “electromigration direction” or “electromigration path” is from the cathode to the focusing zone. In one embodiment, “electromigration direction” or “electromigration path” is from the TE reservoir to the focusing zone. In one embodiment, “electromigration direction” or “electromigration path” is from the LE reservoir to the focusing zone.

In one embodiment, a system as described herein reduces false positive detection. In one embodiment, a system includes means for measuring a signal (such as a photodetector) of a hybrid in at least two time points. In one embodiment, a system includes means for measuring a signal of a hybrid in at least two locations wherein at least one location is situated before the focusing zone (in the electromigration direction). In one embodiment, a photodetector is a photosensor. In one embodiment, a photodetector is a camera. In one embodiment, a photodetector is capable of recording the intensity of a signal derived from a probe such as but not limited to a fluorescence signal. In one embodiment, detecting comprises quantifying. In one embodiment, detect is quantify.

In one embodiment, measuring the hybrid's signal in at least two times points and/or at least two locations during migration of the hybrid-the kinetic process adds significant information for the specific detection of the target. e.g. a high absolute signal at a given time point may be there the result of either a low concentration of matches or a higher concentration of mismatches. In one embodiment, in case the measuring at a later time point or a second point results in a decrease in signal then it originates from a calculatable mismatch, whereas if the signal continues to increase—it is the result of a match.

In one embodiment, a computer program records from a measuring means such as a photodetector, at least two measurements of the hybrid as described herein. In one embodiment, the two or more measurements are applied into algorithms based on the model and equation as described herein which determine the off-rate of the method/process/system.

In one embodiment, a hybrid is a heterohybrid. In one embodiment, a hybrid consists a probe and a nucleic acid molecule-target/analyte. In one embodiment, a hybrid comprises or consists a nucleic acid molecule/labeled-antisense probe mismatch hybrid (false positive). In one embodiment, a hybrid comprises or consists a nucleic acid molecule/labeled-antisense probe match hybrid (true positive). In one embodiment, a hybrid comprises a nucleic acid molecule/labeled-antisense probe match hybrid (true positive) and a nucleic acid molecule/labeled-antisense probe mismatch hybrid (false positive). In one embodiment, a hybrid is a nucleic acid molecule/labeled-antisense probe match hybrid (true positive). In one embodiment, a hybrid is a nucleic acid molecule/labeled-antisense probe mismatch hybrid (false positive).

In one embodiment, provided herein a method for reducing false positive detection of a nucleic acid molecule/labeled-antisense probe mismatch hybrid. In one embodiment, reducing false positive detection of a nucleic acid molecule/labeled-antisense probe mismatch hybrid is increasing the accuracy and/or reliability of the present system and method for detecting and/or quantifying an analyte by ITP. In one embodiment, a probe comprises or consists a labeled-antisense molecule. In one embodiment, a probe is a labeled-antisense molecule. In one embodiment, a probe is a labeled-antisense probe

In one embodiment, the method includes the step of contacting a mixture of nucleic acid molecules with a labeled-antisense molecule (such as but not limited to: PNA, morpholino, nucleic acid molecule) having a sequence of interest in a first solution and obtaining a nucleic acid molecule/labeled-antisense probe mismatch hybrid and a nucleic acid molecule/labeled-antisense probe match hybrid. In one embodiment, a nucleic acid molecule/labeled-antisense probe mismatch hybrid is the result of the binding of a nucleic acid molecule having a sequence which differs by at least one nucleotide from the analyte/target nucleic acid molecule—to the probe. In one embodiment, a nucleic acid molecule/labeled-antisense probe mismatch hybrid is the result of the binding of a nucleic acid molecule having a sequence which differs from the analyte/target nucleic acid molecule—to the probe. In one embodiment, a nucleic acid molecule/labeled-antisense probe mismatch hybrid is the result of the binding of a nucleic acid molecule having a sequence which differs by at least two nucleotides from the analyte/target nucleic acid molecule—to the probe. In one embodiment, a nucleic acid molecule/labeled-antisense probe mismatch hybrid is the result of the binding of a nucleic acid molecule having a sequence which differs by at least three nucleotides from the analyte/target nucleic acid molecule—to the probe. In one embodiment, a nucleic acid molecule/labeled-antisense probe mismatch hybrid is the result of the binding of a nucleic acid molecule having a sequence which differs by at least four nucleotides from the analyte/target nucleic acid molecule—to the probe. In one embodiment, a nucleic acid molecule/labeled-antisense probe match hybrid is the result of the binding of a target nucleic acid molecule/analyte to the probe.

In one embodiment, the method further includes introducing the first solution into the ITP system. In one embodiment, the first solution comprises TE, LE or both. In one embodiment, the method further includes introducing the first solution into the TE. In one embodiment, the method further includes introducing the first solution into the LE. In one embodiment, the method further includes introducing the first solution into a mixture of LE and TE. In one embodiment, the method further includes introducing the first solution between a high effective mobility leading electrolyte and a low effective mobility trailing electrolyte of an isotachophoresis (ITP) system, In one embodiment, the system comprises the first solution positioned or situated in the TE reservoir. In one embodiment, the system comprises the first solution positioned or situated in the LE reservoir. In one embodiment, the system comprises the first solution positioned or situated in a high effective mobility leading electrolyte (LE) reservoir, a low effective mobility trailing electrolyte (TE) reservoir or both.

In one embodiment, the method further includes introducing the first solution in the LE reservoir or within the LE solution. In one embodiment, the method further includes introducing the first solution in the TE reservoir or within the TE solution. In one embodiment, the system is an isotachophoresis (ITP) system. In one embodiment, a second solution comprises or consists LE ions or solution. In one embodiment, a third solution comprises or consists TE ions or solution.

In one embodiment, the system comprises an anode and a cathode. In one embodiment, the method includes applying an electric field across the second solution and the third solution. In one embodiment, the system comprises means for measuring a signal of an electromigrating nucleic acid molecule/labeled-antisense probe mismatch hybrid, an electromigrating nucleic acid molecule/labeled-antisense probe match hybrid (such as a photodetector), or both. In one embodiment, the method comprises measuring a signal of an electromigrating nucleic acid molecule/labeled-antisense probe mismatch hybrid, an electromigrating nucleic acid molecule/labeled-antisense probe match hybrid (such as a photodetector), or both.

In one embodiment, the measuring a signal is measuring at least two signals. In one embodiment, the measuring a signal is measuring at least two signals in at least two different time points.

In one embodiment, measuring a signal is measuring at least two signals in at least two different locations wherein the locations are between the anode and the cathode. In one embodiment, measuring a signal is measuring at least two signals in at least two different locations wherein at least one location is situated before the focusing zone in the direction of the hybrid's electromigration. In one embodiment, measuring a signal is measuring at least two signals in at least two different locations wherein one location is situated before the focusing zone in the direction of the hybrid's electromigration and the second location is situated within the focusing zone. In one embodiment, situated and located includes “captures a signal within the location of”

In one embodiment, measuring a signal is measuring at least two signals at different time points during the migration period of a hybrid and/or at different locations along the migration of a hybrid. In one embodiment, measuring a signal is measuring at least two different time points and/or at least two different locations/distances. In one embodiment, the term “distance” includes “location”. In one embodiment, a first measuring time point (t1) is smaller than the total migration time of a hybrid (Ttotal). In one embodiment, a second measuring time point (t2) is smaller than the total migration time of a hybrid (Ttotal). In one embodiment, a first measuring time point (t1) is smaller than: the second measuring time point (t2), the migration time of a hybrid to the focusing zone (Tfocus), and the total migration time of a hybrid (Ttotal). In one embodiment, a first measuring time point (t2) is equal or smaller to the migration time of a hybrid to the focusing zone (Tfocus). In one embodiment, a first measuring time point (t2) is equal or larger to the migration time of a hybrid to the focusing zone (Tfocus). In one embodiment, a second measuring time point (t2) is larger than t1 and equals to Tfocus. In one embodiment, migration is electromigration.

In one embodiment, measuring a signal is measuring at least two different electromigration distances during the migration period of a hybrid. In one embodiment, measuring a signal is measuring at least two different locations along the electromigration path of a hybrid. In one embodiment, a system or a device comprises photodetectors present in at least two different electromigration distances/locations along the electromigration path of a hybrid.

In one embodiment, at least two different locations along the electromigration path of a hybrid include a first location and a second location. In one embodiment, the first location, the second location, or both is/are located before the focusing zone along the electromigration direction. In one embodiment, the first location, the second location, or both is/are located in-between the anode and the cathode. In one embodiment, the first location is closer to the TE reservoir compared to the second location. In one embodiment, the second location is closer to the focusing zone compared to the first location. In one embodiment, the second location is closer to the LE reservoir compared to the first location.

In one embodiment, along the electromigration path is between the TE solution and the LE solution. In one embodiment, along the electromigration path is between the TE reservoir and the LE reservoir. In one embodiment, along the electromigration path is between the TE solution and the focusing zone. In one embodiment, along the electromigration path is between the TE solution and the focusing zone. In one embodiment, “focusing zone” is within the sharp LE-TE interface. In one embodiment, focusing zone is the location where the hybrid but not the free probe is focused. In one embodiment, focusing zone is the location where the hybrid but not the free analyte is focused.

In one embodiment, a first measuring time point (t1) is smaller than the total migration time of a hybrid (Ttotal). In one embodiment, a second measuring time point (t2) is smaller than the total migration time of a hybrid (Ttotal). In one embodiment, a first measuring time point (t1) is smaller than: the second measuring time point (t2), the migration time of a hybrid to the focusing zone (Tfocus), and the total migration time of a hybrid (Ttotal). In one embodiment, a first measuring time point (t2) is equal or smaller to the migration time of a hybrid to the focusing zone (Tfocus). In one embodiment, a first measuring time point (t2) is equal or larger to the migration time of a hybrid to the focusing zone (Tfocus). In one embodiment, a second measuring time point (t2) is larger than t1 and equals to Tfocus.

in at least two time points or in at least two wherein at least one location is situated before the focusing zone in the electromigration directionc

In one embodiment, the system includes means for measuring at least two signals in at least two different locations wherein the locations are between the anode and the cathode. In one embodiment, means for measuring are located in at least two different locations wherein at least one location is situated before the focusing zone in the direction of the hybrid's electromigration. In one embodiment, the system includes means for measuring at least two signals in at least two different locations wherein one location is situated before the focusing zone in the direction of the hybrid's electromigration and the second location is situated within the focusing zone.

In one embodiment, the system as described herein is set to measure the hybrid at t1, t2. In one embodiment, the system as described herein is set to measure the hybrid at t1, t2 according to Ttotal, Tfocus, or both.

In another embodiment, a system as described herein further comprises a photodetector. In another embodiment, a system as described herein further comprises a photomultiplier tube (PMT). In another embodiment, a system as described herein further comprises a camera. In another embodiment, a system as described herein further comprises a radioactive probe or detector. In another embodiment, a system as described herein further comprises a calorimetric detector.

In another embodiment, a system and a method as described herein detects specific DNA fragments in a sample. In another embodiment, a system and a method as described herein detect specific DNA fragments at a concentration of at least 100 fM (in a sample). In another embodiment, a system and a method as described herein detect specific DNA fragments at a concentration of at least 50 fM (in a sample). In another embodiment, a system and a method as described herein detect specific DNA fragments at a concentration of at least 75 fM (in a sample). In another embodiment, a system and a method as described herein detect specific DNA fragments at a concentration of at least 200 fM (in a sample). In another embodiment, a system and a method as described herein detect specific DNA fragments at a concentration of at least 500 fM (in a sample). In another embodiment, a system and a method as described herein detect as little as 75 fM of specific DNA fragments in a sample. In another embodiment, a system and a method as described herein detect as little as 100 fM of specific DNA fragments in a sample. In another embodiment, a system and a method as described herein detect as little as 200 fM of specific DNA fragments in a sample. In another embodiment, a system and a method as described herein detect as little as 500 fM of specific DNA fragments in a sample.

In another embodiment, a system and a method as described herein demonstrate 5 orders of magnitude dynamic range. In another embodiment, a system and a method as described herein demonstrate 2-8 orders of magnitude dynamic range. In another embodiment, a system and a method as described herein demonstrate 3-10 orders of magnitude dynamic range.

In another embodiment, the present invention provides an ITP kit comprising a morpholino molecule for probing a nucleic acid molecule having a specific sequence of interest and specific instructions for preparing a TE buffer and a LE buffer. In another embodiment, the present invention provides an ITP kit comprising a system of the invention. In another embodiment, the present invention provides a kit comprising an instruction manual describing the method and/or system disclosed herein.

In another embodiment, probing is sequence specific probing utilizing at least one morpholino molecule as described herein. In another embodiment, probing is probing a sequence as short as 7 contiguous nucleic acid residues (such as DNA or RNA). In another embodiment, probing is probing a sequence comprising at least 7 contiguous nucleic acid residues (such as DNA or RNA). In another embodiment, probing is sequence specific probing. In another embodiment, probing is probing a sequence comprising 10 or more contiguous nucleic acid residues . In another embodiment, probing is sequence specific probing. In another embodiment, probing is probing at least a sequence consisting 10 to 1000 contiguous nucleic acid residues. In another embodiment, probing is probing at least a sequence consisting 10 to 500 contiguous nucleic acid residues. In another embodiment, probing is also a measure of the number “n” provided in the morpholino formula.

In another embodiment, the present invention provides a kit for carrying out ITP separation of nucleic acid analytes/targets in a sample. According to some embodiments of the present invention, wherein the kit comprises LE buffer, TE buffer, and a morpholino molecule for sequence specific identification and/or isolation of a nucleic acid molecule of interest.

In another embodiment, the present invention provides a kit as described herein further comprising an electrophoresis apparatus. In another embodiment, the present invention provides a kit as described herein further comprising an electrophoresis apparatus coupled to a central processing unit (CPU) that may operate the electrophoresis apparatus based on a predetermined set of instructions. In another embodiment, the present invention provides a kit further comprising the target nucleic acid as a positive control. In another embodiment, the present invention provides a kit further comprising a negative control comprising a sequence having at least a single nucleic acid addition, deletion, or substitution compared to the target nucleic acid molecule.

In another embodiment, the invention further provides a method for sequence-specifically detecting a nucleic acid molecule, using the system as described herein and comprising the steps of: (a) introducing the morpholino probe with nucleic acid sample into the ITP system (such as but not limited into the TE solution, LE solution or a mixture of TE and LE), obtaining a nucleic acid molecule/morpholino hybrid ; applying electrical field in the ITP system or between the LE and TE zones such that the nucleic acid molecule/morpholino hybrid but not free morpholino focus at the sharp LE-TE interface enabling detection of the nucleic acid molecule/morpholino hybrid; (b) injecting a finite volume of a mixture composed of a nucleic acid molecule/morpholino hybrid into the TE and/or LE ; applying electrical field between the LE and TE zones such that the nucleic acid molecule/morpholino hybrid but not free morpholino focus at the sharp LE-TE interface enabling detection of the nucleic acid molecule/morpholino hybrid; (c) injecting a finite volume of a mixture composed of morpholino probes into the LE solution or the TE solution and the nucleic acid sample into the LE solution or the TE solution; applying electrical field between the LE and TE zones such that a high concentration zone of nucleic acid molecule/morpholino hybrid is formed at the sharp LE-TE interface enabling detection of the nucleic acid molecule/morpholino hybrid.

In one embodiment, the reagents, system and kits of the invention enable a method for separating and/or detecting a match hybrid. In one embodiment, provided a method for separating and/or detecting a match hybrid comprising a first molecule and a second molecule, comprising the steps of: contacting a mixture of molecules comprising the first molecule and the second molecule in a first solution and obtaining hybrids; introducing the first solution into the ITP system, the ITP system comprises a second solution of high effective mobility leading electrolyte (LE) ions and a third solution of low effective mobility trailing electrolyte (TE); applying voltage between the LE and the TE, inducing a low electrical field in the leading electrolyte and a high electrical field in the terminating electrolyte; wherein only the hybrids focus at the sharp LE-TE interface in the ITP system, thereby separating and/or detecting/quantifying a specific hybrid.

In one embodiment, the reagents, system and kits of the invention enable a method for separating and/or detecting a match hybrid. In one embodiment, provided a method for separating and/or detecting a match hybrid comprising a first molecule and a second molecule, comprising the steps of: contacting a mixture of molecules comprising the first molecule and the second molecule in a first solution and obtaining hybrids; introducing the first solution into the ITP system, the ITP system comprises a second solution of high effective mobility leading electrolyte (LE) ions and a third solution of low effective mobility trailing electrolyte (TE); applying voltage between the LE and the TE, inducing a low electrical field in the leading electrolyte and a high electrical field in the terminating electrolyte; wherein only: (A) the hybrids; and/or (B) the hybrids and first molecule focus at the sharp LE-TE interface in the ITP system, thereby separating and/or detecting/quantifying a specific hybrid.

In one embodiment, a match hybrid comprises or consists two molecules that specifically interact. In one embodiment, a match hybrid comprises or consists two molecules bound due to structural compatibility. In one embodiment, a match hybrid is formed by molecular binding between two molecules that results in a stable association in which the molecules are in close proximity to each other. In one embodiment, a match hybrid comprises a molecular bond between the two molecules (a first and a second molecule). In one embodiment, a match hybrid comprises a non-covalent bond between the two molecules (a first and a second molecule). In one embodiment, a match hybrid comprises a reversible covalent bond between the two molecules (a first and a second molecule). In one embodiment, a match hybrid comprises an irreversible covalent bond between the two molecules (a first and a second molecule).

In one embodiment, provided a method for separating and/or detecting a match hybrid is a ligand binding assay. In one embodiment, a match hybrid comprises a ligand molecule and a receptor. In one embodiment, a match hybrid comprises a ligand molecule and an antibody. In one embodiment, a match hybrid comprises a ligand molecule and a target macromolecule. In one embodiment, a match hybrid comprises a ligand molecule and a target molecule. In one embodiment, a match hybrid comprises an enzyme and the enzyme's substrate (a first and a second molecule). In one embodiment, a match hybrid comprises an antibody and the antibody's antigen (a first and a second molecule).

In one embodiment, hybrids comprise match and mismatch hybrids. In one embodiment, a match hybrid and mismatch hybrids. In one embodiment, the binding constant (KA) between a first molecule and a second molecule within a match hybrid is higher than the Ka of a mismatch hybrid. In one embodiment, the binding affinity between a first molecule and a second molecule within a match hybrid is higher than the binding affinity of a mismatch hybrid. In one embodiment, the ΔG⁰_{bind} of a match hybrid at 300K (kJmol⁻¹) is at least −10. In one embodiment, the ΔG⁰_{bind} of a match hybrid at 300K (kJmol⁻¹) is at least −15. In one embodiment, the ΔG⁰_{bind} of a match hybrid at 300K (kJmol⁻¹) is at least −20. In one embodiment, the ΔG⁰_{bind} of a match hybrid at 300K (kJmol⁻¹) is at least −10. In one embodiment, the ΔG⁰_{bind} of a match hybrid at 300K (kJmol⁻¹) is at least −30. In one embodiment, the ΔG⁰_{bind} of a match hybrid at 300K (kJmol⁻¹) is at least −40. In one embodiment, the ΔG⁰_{bind} of a match hybrid at 300K (kJmol⁻¹) is at least −50. In one embodiment, the ΔG⁰_{bind} of a match hybrid at 300K (kJmol⁻¹) is at least −60.

In one embodiment, the ΔG⁰_{bind} of a mismatch hybrid at 300K (kJmol⁻¹) is less than −10. In one embodiment, the ΔG⁰_{bind} of a mismatch hybrid at 300K (kJmol⁻¹) is less than −15. In one embodiment, the ΔG⁰_{bind} of a mismatch hybrid at 300K (kJmol⁻¹) is less than −20. In one embodiment, the ΔG⁰_{bind} of a mismatch hybrid at 300K (kJmol⁻¹) is less than −30. In one embodiment, the ΔG⁰_{bind} of a match hybrid at 300K (kJmol⁻¹) is at least 20% greater than the less than ΔG⁰_{bind} of a mismatch hybrid at 300K (kJmol⁻¹). In one embodiment, the ΔG⁰_{bind} of a match hybrid at 300K (kJmol⁻¹) is at least 25% greater than the less than ΔG⁰_{bind} of a mismatch hybrid at 300K (kJmol⁻¹). In one embodiment, the ΔG⁰_{bind} of a match hybrid at 300K (kJmol⁻¹) is at least 50% greater than the less than ΔG⁰_{bind} of a mismatch hybrid at 300K (kJmol⁻¹). In one embodiment, the ΔG⁰_{bind} of a match hybrid at 300K (kJmol⁻¹) is at least 75% greater than the less than ΔG⁰_{bind} of a mismatch hybrid at 300K (kJmol⁻¹). In one embodiment, the ΔG⁰_{bind} of a match hybrid at 300K (kJmol⁻¹) is at least 100% greater than the less than ΔG⁰_{bind} of a mismatch hybrid at 300K (kJmol⁻¹). In one embodiment, the ΔG⁰_{bind} of a match hybrid at 300K (kJmol⁻¹) is at least 150% greater than the less than ΔG⁰_{bind} of a mismatch hybrid at 300K (kJmol⁻¹). In one embodiment, the Δ⁰_{bind} of a match hybrid at 300K (kJmol⁻¹) is at least 200% greater than the less than ΔG⁰_{bind} of a mismatch hybrid at 300K (kJmol⁻¹).

In one embodiment, a first molecule is a ligand such as a small molecule and a second molecule which is a protein. In one embodiment, a first molecule and a second molecule within a hybrid are allosterically bound. In one embodiment, a first molecule and a second molecule within a hybrid are non-allosterically bound.

In one embodiment, the hybrid is a dimer. In one embodiment, the hybrid is a heterodimer. In one embodiment, the hybrid is a homodimer. In one embodiment, the first molecule and the second molecule are identical. In one embodiment, the first molecule is labeled. In one embodiment, the second molecule is labeled.

In one embodiment, provided a method for sequence-specifically detecting and/or separating a nucleic acid molecule, comprising the steps of: contacting a mixture of nucleic acid molecules with a morpholino having a sequence of interest in a first solution and obtaining hybrids; introducing the first solution comprising the hybrids resulting from step (a) into an ITP system, the ITP system comprises a second solution of high effective mobility leading electrolyte (LE) ions, a third solution of low effective mobility trailing electrolyte (TE), and a fourth solution of intermediate effective mobility electrolyte (IE); applying an electric field across the second solution and the third solution; wherein the hybrid but not free morpholino focus at the sharp LE-IE interface in the ITP system and the free morpholino but not the hybrids focus at the sharp IE-TE interface in the ITP system, wherein the IE has a higher mobility than the morpholino probe and the IE has a lower mobility than the hybrid, thereby sequence-specifically separating a nucleic acid molecule.

In one embodiment, a hybrid comprises a sense nucleic acid molecule and an antisense nucleic acid molecule. In one embodiment, a first molecule is a sense nucleic acid molecule and the second molecule is an antisense nucleic acid molecule. In one embodiment, “morpholino” according to the invention is interchangeable with a first molecule. In one embodiment, “morpholino” according to the invention is interchangeable with a second molecule. In one embodiment, “ nucleic acid molecule of interest” according to the invention is interchangeable with a first molecule. In one embodiment, “In one embodiment, “morpholino” according to the invention is interchangeable with a first molecule. In one embodiment, “morpholino” according to the invention is interchangeable with a second molecule” according to the invention is interchangeable with a second molecule. In one embodiment, “antisense” according to the invention is interchangeable with a first molecule. In one embodiment, “antisense” according to the invention is interchangeable with a second molecule

In another embodiment, a method for separating and/or isolating a nucleic acid molecule of interest, the morpholino, or the hybrid consisting the morpholino/nucleic acid molecule of interest, further comprises subjecting the ITP focused composition which comprises free nucleic acid molecules and hybrids of morpholino/nucleic acid molecule (but free of unhybridized morpholinos) to a second step that actually separates and/or isolates the nucleic acid molecule of interest and/or the hybrid comprising morpholino/nucleic acid molecule of interest. In another embodiment, the second step includes subjecting the composition which comprises free nucleic acid molecules and hybrids of morpholino/nucleic acid molecule to an electric field and separating the hybrid according to its isoelectric point. In another embodiment, the second step includes subjecting the composition which comprises free nucleic acid molecules and hybrids of morpholino/nucleic acid molecule to a separating column which is capable of separating/distinguishing free nucleic acid molecules from hybrids of the invention. In another embodiment, an electric field is applied across the ITP solutions or buffers. In another embodiment, an electric field is applied across the TE and LE solutions/buffers.

In another embodiment, a third step of separating the nucleic acid of interest from the morpholino is applied. In another embodiment, the third step includes subjecting the hybrid to urea or any other solution capable of separating the morpholino-nucleic acid molecule. In another embodiment, a fourth step of discarding the morpholino is applied. In another embodiment, methods of isolating a morpholino or a nucleic acid molecule from a solution that comprises both morpholino and a nucleic acid molecule are known to one of skill in the art. In another embodiment, the methods of the present invention result in separating, isolating and/or enriching the nucleic acid molecule of interest or the morpholino.

In another embodiment, the invention further provides a method for sequence-specifically separating or isolating a nucleic acid molecule, comprising the steps of: (a) contacting a mixture of nucleic acid molecules with a labeled morpholino having a sequence of interest in a first solution and obtaining a nucleic acid molecule/morpholino hybrid (sense-anti-sense complex); (b) introducing the first solution into the TE, into the LE, into a mixture of LE and TE, or between a high effective mobility leading electrolyte and a low effective mobility trailing electrolyte of an isotachophoresis (ITP) system, the ITP system comprises a second solution of high effective mobility leading electrolyte (LE) ions and a third solution of low effective mobility trailing electrolyte (TE); and (c) applying a low electrical field in the leading electrolyte and a high electrical field in the terminating electrolyte; wherein the nucleic acid molecule/morpholino hybrid but not free morpholino focus at the sharp LE-TE interface in the ITP system, thereby sequence-specifically separating a nucleic acid molecule.

In another embodiment, the first solution has an ionic strength that enables stringent hybridization conditions between the morpholino and the target nucleic acid molecule. In another embodiment, a person of ordinary skill in the art can readily prepare a solution that enables stringent hybridization conditions as described herein. In another embodiment, the nucleic acid molecule is 10-100 nucleotides long and the morpholino molecule renders the hybrid positively charged. In another embodiment, the nucleic acid molecule is 10-50 nucleotides long and the morpholino molecule renders the hybrid positively charged (wherein the unhybridized nucleic acid molecules are negatively charged).

In another embodiment, morpholino having a sequence of interest is a morpholino directed against a particular sequence of DNA or RNA. In another embodiment, morpholino having a sequence of interest is a morpholino that will bind only a DNA or a RNA molecule comprising the sequence of interest to which the morpholino molecule is designed a hybridize to. In another embodiment, morpholino having a sequence of interest is a morpholino that specifically binds a particular sequence of DNA or RNA under stringent conditions.

In another embodiment, the phrase “sequence-specifically separating” includes the phrase “sequence-specifically identifying”. In another embodiment, a morpholino probe as described herein is further labeled as described hereinabove. In another embodiment, the term “isolating” is substituted with the term “enriching”. In another embodiment, separating is discarding the free unhybridized morpholino.

In another embodiment, the present invention provides methods, systems and kits that reduce false positive or false negative results. In another embodiment, the present invention provides methods, systems and kits that reduce background noise. In another embodiment, the present invention provides methods, systems and kits that reduce background originating from a free morpholino molecule or false identification of the target nucleic acid molecule. In another embodiment, the present invention provides methods, systems and kits that provide accurate quantitative measures of the nucleic acid molecule of interest. In another embodiment, the present invention provides methods, systems and kits that provide an efficient separating technique for a nucleic acid molecule of interest. In another embodiment, the present invention provides methods wherein the free, unhybridized, morpholinos aren't focused in the interface. In another embodiment, free morpholinos aren't focused in the interface. In another embodiment, the method and system of the invention includes at least one detection unit for detecting the label of the morpholino probe. In another embodiment, detection units for detecting different labels are known to one of average skill in the art. In another embodiment, detection units for detecting different labels are described hereinabove.

In another embodiment, “labeled” comprises a label. In another embodiment, the label is Acridine orange. In another embodiment, the label is Acridine yellow. In another embodiment, the label is Alexa Fluor. In another embodiment, the label is 7-Aminoactinomycin D. In another embodiment, the label is 8-Anilinonaphthalene-1-sulfonic acid. In another embodiment, the label is an ATTO dye. In another embodiment, the label is Auramine-rhodamine stain. In another embodiment, the label is Benzanthrone. In another embodiment, the label is Bimane. In another embodiment, the label is 9,10-Bis(phenylethynyl)anthracene. In another embodiment, the label is 5,12-Bis(phenylethynyl)naphthacene. In another embodiment, the label is Bisbenzimide. In another embodiment, the label is a Blacklight paint. In another embodiment, the label is Brainbow. In another embodiment, the label is Calcein. In another embodiment, the label is Carboxyfluorescein. In another embodiment, the label is Carboxyfluorescein diacetate succinimidyl ester. In another embodiment, the label is Carboxyfluorescein succinimidyl ester. In another embodiment, the label is 1-Chloro-9,10-bis(phenylethynyl)anthracene. In another embodiment, the label is 2-Chloro-9,10-bis(phenylethynyl)anthracene. In another embodiment, the label is 2-Chloro-9,10-diphenylanthracene. In another embodiment, the label is Coumarin. In another embodiment, the label is DAPI. In another embodiment, the label is a Dark quencher. In another embodiment, the label is DiOC6. In another embodiment, the label is DyLight Fluor. In another embodiment, the label is Ethidium bromide. In another embodiment, the label is Fluo-3.

In another embodiment, the label is Fluo-4. In another embodiment, the label is a FluoProbe. In another embodiment, the label is Fluorescein. In another embodiment, the label is Fluorescein isothiocyanate. In another embodiment, the label is a Fluoro-Jade stain. In another embodiment, the label is Fura-2. In another embodiment, the label is Fura-2-acetoxymethyl ester. In another embodiment, the label is GelGreen. In another embodiment, the label is GelRed. In another embodiment, the label is Green fluorescent protein. In another embodiment, the label is a Heptamethine dye. In another embodiment, the label is Hoechst stain. In another embodiment, the label is Indian yellow. In another embodiment, the label is Indo-1. In another embodiment, the label is Lucifer yellow. In another embodiment, the label is Luciferin. In another embodiment, the label is MCherry. In another embodiment, the label is Merocyanine. In another embodiment, the label is Nile blue. In another embodiment, the label is Nile red. In another embodiment, the label is an Optical brightener. In another embodiment, the label is Perylene. In another embodiment, the label is Phloxine. In another embodiment, the label is P cont. In another embodiment, the label is Phycobilin. In another embodiment, the label is Phycoerythrin. In another embodiment, the label is Phycoerythrobilin. In another embodiment, the label is Propidium iodide. In another embodiment, the label is Pyranine. In another embodiment, the label is a Rhodamine. In another embodiment, the label is RiboGreen. In another embodiment, the label is RoGFP. In another embodiment, the label is Rubrene. In another embodiment, the label is (E)-Stilbene. In another embodiment, the label is (Z)-Stilbene. In another embodiment, the label is a Sulforhodamine. In another embodiment, the label is SYBR Green I. In another embodiment, the label is Synapto-pHluorin. In another embodiment, the label is Tetraphenyl butadiene. In another embodiment, the label is Tetrasodium tris(bathophenanthroline disulfonate)ruthenium(II). In another embodiment, the label is Texas Red. In another embodiment, the label is Titan yellow. In another embodiment, the label is TSQ. In another embodiment, the label is Umbelliferone. In another embodiment, the label is Yellow fluorescent protein. In another embodiment, the label is YOYO-1. In another embodiment, the label is a chemiluminescent dye. In another embodiment, the label is a radioisotope or a radioactive dye. In another embodiment, the label is a dye that can be detected by a naked eye.

In another embodiment, the nucleic acid molecule which is the target sequence to be detected by specific hybridization comprises 7 to 10000 bases. In another embodiment, the nucleic acid molecule which is the target sequence to be detected comprises 10 to 10000 bases. In another embodiment, the nucleic acid molecule which is the target sequence to be detected comprises 10 to 1000 bases. In another embodiment, the nucleic acid molecule which is the target sequence to be detected comprises 10 to 500 bases. In another embodiment, the nucleic acid molecule which is the target sequence to be detected comprises 20 to 400 bases. In another embodiment, the nucleic acid molecule which is the target sequence to be detected comprises 50 to 500 bases. In another embodiment, the nucleic acid molecule which is the target sequence to be detected comprises 100 to 10000 bases. In another embodiment, the nucleic acid molecule which is the target sequence to be detected comprises 500 to 5000 bases.

In another embodiment, the nucleic acid molecule/morpholino hybrid has a higher mobility than the TE. In another embodiment, the nucleic acid molecule/morpholino hybrid has a lower mobility than the LE. In another embodiment, the TE has a higher mobility than the free morpholino probe. In another embodiment, the TE has a lower mobility than the nucleic acid molecule/morpholino hybrid. In another embodiment, the TE has a lower mobility than the nucleic acid molecule.

In another embodiment, the first solution comprises TE. In another embodiment, the first solution comprises LE.

In another embodiment, “morpholino” is non-labeled morpholino. In another embodiment, “morpholino” is a labeled morpholino. In another embodiment, labeled morpholino comprises a single sequence of morpholino directed against one specific nucleic acid sequence. In another embodiment, labeled morpholino comprises multiple sequences of morpholinos directed against multiple nucleic acid sequences wherein each morpholino is labeled with a different molecule or dye. In another embodiment, each morpholino (having a specific sequence) is designed to induce a specific charge on the hybrid thus each hybrid is distinguishable according to its unique electrical charge.

In another embodiment, the term “hybrid” is defined as a complex of morpholino with DNA, RNA, or a molecule comprising both DNA and RNA bases. In another embodiment, “hybrid” is defined as a complex depending on base pairing. In another embodiment, the term “hybrids” includes a match hybrid and a mismatch hybrid. In another embodiment, the term “hybrids” consists match hybrid. In another embodiment, the term “hybrids” consists mismatch hybrid.

In another embodiment, the target nucleic acid molecule to be probed and morpholino are mixed with the LE and/or the TE. In another embodiment, the effective mobility of the “hybrid” (the bound heterocomplex) and the effective mobility of morpholino differ.

In another embodiment, bound complex/hybrid is translocated to and/or extracted from ITP focus zone. In another embodiment, analysis of the bound complex is further performed in the ITP zone of the hybrid.

In another embodiment, the method of the present invention can be utilized to identify a nucleic acid molecule of interest such as a nucleic acid molecule that identifies a pathogen. In another embodiment, the method of the present invention can be utilized to identify a nucleic acid molecule of interest such as a gene of interest or a regulatory element of interest. In another embodiment, the method of the present invention can be utilized for screening an infection by obtaining a patient specimen (e.g., urine sample, blood sample etc.), and performing the above described methods and analyses, where the ITP sample is derived from the patient specimen, and the morpholino is capable of binding to the target nucleic acid molecule which is a marker for disease. In another embodiment, nucleic acid molecules that can serve as markers for disease include bacterial nucleotide sequences, viral RNA or DNA sequences, mitochondrial DNA sequences, micro RNA sequences, or messenger RNA sequences that encode host or pathogen proteins involved in disease, etc. In one embodiment, a nucleic acid molecule comprises or consists a sequence of interest. In one embodiment, the antisense comprises or consists a sequence of interest. In one embodiment, the antisense comprises or consists a sequence of interest which specifically binds to the sequence of interest of the nucleic acid molecule. In one embodiment, the antisense comprises or consists a sequence of interest which specifically binds via base-pairing to the sequence of interest of the nucleic acid molecule. In one embodiment, the antisense specifically binds to the sequence of interest of the nucleic acid molecule. In one embodiment, the antisense preferably binds to the sequence of interest of the nucleic acid molecule under stringent conditions as described herein.

In one embodiment, the target nucleic acid molecule comprises or consists a sequence of interest. In one embodiment, the antisense/probe specifically binds to the sequence of interest under stringent conditions. In one embodiment, the sequence of interest within the antisense/probe specifically binds to the sequence of interest within the nucleic acid sequence, under stringent conditions. In one embodiment, the sequence of interest within the antisense/probe forms base-pairing with the sequence of interest within the nucleic acid sequence, under stringent conditions.

In one embodiment, a nucleic acid molecule is the target sequence or the target nucleic acid molecule. In one embodiment, the sequence is at least 4 bases long. In one embodiment, the sequence is at least 5 bases long. In one embodiment, the sequence is at least 6 bases long. In one embodiment, the sequence is at least 8 bases long. In one embodiment, the sequence is at least 10 bases long. In one embodiment, the sequence is at least 12 bases long. In one embodiment, the sequence is 4 to 400 bases long. In one embodiment, the sequence is at least 12 bases long. In one embodiment, the sequence is 6 to 200 bases long. In one embodiment, the sequence is at least 12 bases long. In one embodiment, the sequence is 6 to 100 bases long. In one embodiment, the sequence is at least 12 bases long. In one embodiment, the sequence is 8 to 50 bases long.

In another embodiment, the present method requires minimal sample preparation and performs extraction, focusing, and detection of a target nucleic acid molecule in a single step.

In another embodiment, the theory behind ITP is provided in Bahga S S, Kaigala G V, Bercovici M, Santiago J G. High-sensitivity detection using isotachophoresis with variable cross-section geometry. Electrophoresis. 2011 Feb;32(5):563-72; Khurana T K, Santiago J G. Sample zone dynamics in peak mode isotachophoresis. Anal Chem. 2008 Aug 15;80(16):6300-7; and Isotachophoresis: Theory, Instrumentation and Applications. F. M. Everaerts, J. L. Beckers, T.P.E.M. Verheggen, Elsevier, Sep. 22, 2011, which are hereby incorporated by reference in their entirety.

In another embodiment, ITP is performed in a peak mode. In another embodiment, ITP is performed in a plateau mode. In another embodiment, “Plateau mode” refers to a wide sample-zone compared to the transition zones, i.e. the sample concentration distribution forms a plateau with blurred boundaries towards LE and TE. In another embodiment, “Peak mode” refers to a very short sample zone, where the two transition zones at both sides of the sample overlap or when the sample is entirely within the interface between LE and TE. In another embodiment, a sample comprises a hybrid composed of morpholino and a nucleic acid molecule.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Strategies for Protein Purification and Characterization—A Laboratory Course Manual” CSHL Press (1996); all of which are incorporated by reference. Other general references are provided throughout this document.

### Materials and Methods

An ITP system was used for the current experiments. The system included a simple microchannel connected to two reservoirs that were initially filled with LE solution. (a) Analytes (the target DNA sequences) were mixed in the trailing electrolyte (TE) reservoir. (b) When an electric field was applied all ions electromigrate in the channel. The LE and TE were chosen such that analytes of interest had a higher mobility than the TE, but cannot over-speed—the LE. These settings result in selective focusing at the sharp LE-TE interface.

### Study Design

Two with morpholino backbones (GeneTools, Philomath, Oreg., USA) were used. both morpholino probes were labeled with lissamine. The morpholino probes targeted the E. coli-specific 16S rRNA sequence, and the DNA targets used with these probes either corresponded to this sequence identically, or included 1-5 non-complementary (“mismatched”) bases in the region complementary to the probe.

2-(N-morpholino)ethanesulfonic acid (MES), 2,2-Bis(hydroxymethyl)-2,2′, 2″-nitrilotriethanol (BISTRIS), and 1.3 MDa polyvinylpyrrolidone (PVP) were purchased from Sigma-Aldrich Israel (Rehovot, Israel), hydrochloric acid (HCl) from Merck (Darmstadt, Germany), and sodium hydroxide (NaOH) from BioLab (Jerusalem, Israel). Morpholino oligos modified with Lissamine fluorophore were purchased from GeneTools (Philomath, Oreg., USA); These oligos were designed to hybridize with a conserved sequence of E. coli 16S rRNA. Reconstituted Morpholino oligos were stored in DI, and stored the stock solutions at room temperature in a humidity chamber. At the beginning of every working day, Morpholino stock was vortexed and heated to 65° C. using a mini dry bath incubator (QSR Technologies, Markham, Ontario, Canada) for 10 min. These 80-nucleotide (nt) synthetic DNA sequences were purchased from Sigma-Aldrich Israel (Rehovot, Israel). These DNA oligonucleotides were reconstituted in DI, and stored these oligo stock solutions at −20° C. in 5-10 μL aliquots.

An additional DNA sequence that had only a 4 nt-long sequence similarity to the E. coli probes was used to represent a randomly-binding DNA sequence. All DNA sequences were purchased from Sigma-Aldrich Israel (Rehovot, Israel).

The set of three E. coli-specific probes consisted of one 14 nt-long morpholino probe, and a 25-nt long morpholino probe (Tables 1 and 2 also list figures in which each sequence was used. The presence of the “CTTA” sequence, in bold, which is not included in sequencing data and therefore does not match the 25 nt probe.

Table 1). The sequence of the 25 nt morpholino probe was taken from E. coli sequencing data. Several sets of fully complementary and mismatch DNA sequences, all 80 nt long, were used as targets. In the first set (Set 1, Table 2), the sequence for the 80 nt-long matching and list of figures in which each was used. Shading shows bases present in 14 nt probe; underlining shows bases present in 25 nt probe. Tables 1 and 2 also list figures in which each sequence was used. The presence of the “CTTA” sequence, in bold, which is not included in sequencing data and therefore does not match the 25 nt probe.

TABLE 1
E. coli-specific probes is provided in FIG. 14

| Morpholino—14 nt | CGTCAATGAGCAAA-Lissamine | FIG. |
|---|---|---|
| | (SEQ ID NO: 1) | 3 |
| Morpholino—25 nt | CTTCTGCGGGTAACGTCAATGAGCA- | FIGS. |
| | (SEQ ID NO: 2) Lissamine | 2-5 |

TABLE 2
Set 1 of E. coli-based DNA targets, for
use with 14 nt probe is provided in FIG. 15.

| | |
|---|---|
| Match | GAGTAAAGTTAATACCTTTGCTCATTGACGTTACTT |
| | ACCCGCAGAAGAAGCACCGGCTAACTCCGTGCCAGC |
| | AGCCGCGG (SEQ ID NO: 3) |
| 1 nt | GAGTAAAGTTAATACCTTTGCTTATTGACGTTACTT |
| mismatch | ACCCGCAGAAGAAGCACCGGCTAACTCCGTGCCAGC |
| | AGCCGCGG (SEQ ID NO: 4) |
| 2 nt | GAGTAAAGTTAATACCTTTGCTTGTTGACGTTACTT |
| mismatch | ACCCGCAGAAGAAGCACCGGCTAACTCCGTGCCAGC |
| | AGCCGCGG (SEQ ID NO: 5) |
| 3 nt | GAGTAAAGTTAATACCTTTGCTGGATGACGTTACTT |
| mismatch | ACCCGCAGAAGAAGCACCGGCTAACTCCGTGCCAGC |
| | AGCCGCGG (SEQ ID NO: 6) |
| 4 bp | GAGTAAAGTTAATACCTTTGCGACGTGACGTTACTT |
| mismatch | ACCCGCAGAAGAAGCACCGGCTAACTCCGTGCCAGC |
| | GACCGCGG (SEQ ID NO: 7) |
| 5 bp | GAGTAAAGTTAATACCTTTGCGACGCGACGTTACTT |
| mismatch | ACCCGCAGAAGAAGCACCGGCTAACTCCGTGCCAGC |
| | AGCCGCGG (SEQ ID NO: 8) |

TABLE 3
(provided in FIG. 16). Set 2 of E. coli-
based DNA targets, for use with 25 nt probe,
and list of figures in which each was used.

| | | |
|---|---|---|
| Match | AGGAAGGGAGTAAAGTTAATACCTTT | FIGS. |
| | GCTCATTGACGTTACCCGCAGAAGAA | 2-5 |
| | GCACCGGCTAACTCCGTGCCAGCAGC | |
| | CG (SEQ ID NO: 9) | |
| 1 nt MM | AGGAAGGGAGTAAAGTTAATACCTTT | |
| (L = 25 nt) | GCTCATTGACGATACCCGCAGAAGAA | |
| Match | GCACCGGCTAACTCCGTGCCAGCAGC | |
| (L = 14 nt) | CG (SEQ ID NO: 10) | |
| 2 nt MM | AGGAAGGGAGTAAAGTTAATACCTTT | |
| (L = 25 nt) | GCTCATTGACGAGACCCGCAGAAGAA | |
| Match | GCACCGGCTAACTCCGTGCCAGCAGC | |
| (L = 14 nt) | CG (SEQ ID NO: 11) | |
| 3 nt MM | AGGAAGGGAGTAAAGTTAATACCTTT | |
| (L = 25 nt) | GCTCATTGACTAGACCCGCAGAAGAA | |
| 1 nt MM | GCACCGGCTAACTCCGTGCCAGCAGC | |
| (L = 14 nt) | CG (SEQ ID NO: 12) | |
| 4 nt MM | AGGAAGGGAGTAAAGTTAATACCTTT | FIG. |
| (L = 25 nt) | GCTCATTGACTAGCCCCGCAGAAGAA | 3 |
| 1 nt MM | GCACCGGCTAACTCCGTGCCAGCAGC | |
| (L = 14 nt) | CG (SEQ ID NO: 13) | |
| 5 nt MM | AGGAAGGGAGTAAAGTTAATACCTTT | FIGS. |
| (L = 25 nt) | GCTCATTGATTAGCCCCGCAGAAGAA | 2-3 |
| 2 nt MM | GCACCGGCTAACTCCGTGCCAGCAGC | |
| (L = 14 nt) | CG (SEQ ID NO: 14) | |
| Random | CTCAGAGTATATACATTCCATAGATC | FIGS. |
| | TGGATACCCGTGACGAATAAAGATCA | 2-5 |
| | GAGTATATACATTCCATAGATCTGCA | |
| | TA (SEQ ID NO: 15) | |

Bases complementary to the 14 nt probe are shaded and bases complementary to the 25 nt probe are underlined.
MM = mismatch, L = probe length.
Random sequence contains 4-nt binding stretch “CATT”, which is complementary to both probes.

TABLE 4
(provided in FIG. 17). Set 3 of E. coli-based
DNA targets, for use with 14 nt and 25 nt probes.

| | |
|---|---|
| Match | GAGTAAAGTTAATACCTTTGCTCATTGACGTTACCC |
| | GCAGAAGAAGCACCGGCTAACTCCGTGCCAGCAGCC |
| | GCGGTAAT (SEQ ID NO: 16) |
| 1 nt | GAGTAAAGTTAATACCTTTGCTTATTGACGTTACCC |
| mismatch | GCAGAAGAAGCACCGGCTAACTCCGTGCCAGCAGCC |
| | GCGGTAAT (SEQ ID NO: 17) |
| 2 nt | GAGTAAAGTTAATACCTTTGCTTGTTGACGTTACCC |
| mismatch | GCAGAAGAAGCACCGGCTAACTCCGTGCCAGCAGCC |
| | GCGGTAAT (SEQ ID NO: 18) |
| 3 nt | GAGTAAAGTTAATACCTTTGCTGGATGACGTTACCC |
| mismatch | GCAGAAGAAGCACCGGCTAACTCCGTGCCAGCAGCC |
| | GCGGTAAT (SEQ ID NO: 19) |
| 4 nt | GAGTAAAGTTAATACCTTTGCGACGTGACGTTACCC |
| mismatch | GCAGAAGAAGCACCGGCTAACTCCGTGCCAGCAGCC |
| | GCGGTAAT (SEQ ID NO: 20) |
| 5 nt | GAGTAAAGTTAATACCTTTGCGACGCGACGTTACCC |
| mismatch | GCAGAAGAAGCACCGGCTAACTCCGTGCCAGCAGCC |
| | GCGGTAAT (SEQ ID NO: 21) |

Bases complementary to the 14 nt probe are shaded and bases complementary to the 25 nt probe are underlined.
These targets were not used in experiments presented here

When designing this second set of targets, the location of the mismatched bases was also changed to be closer to the center of the region complementary to the 25-nt probe. However, this shift caused the Set 2 targets to be partially incompatible with the 14 nt probes, as some of the mismatches were now outside the region complementary to the 14 nt probe, causing a 5-nt mismatch to the 25-nt probe to be only a 2-nt mismatch to the 14-nt probe. To address this issue, another set of probes (Set 3, Table 4) was designed, in which all of the mismatches were within the region that overlapped with both the 14 nt and the 25 nt probes, and the CTTA sequence was absent, allowing this final set of targets to be used with any of the E. coli-specific probes. This set of targets was not used in obtaining the results presented here; however, it is included here for the benefit of the reader.

An additional 80 nt DNA sequence was designed to model “random” binding with only n =4 binding bases. The sequence (included in Set 2 of the targets) contains two instances of the 4 nt sequence “CATT”, which is complementary to all of the E. coli-specific probes, but contains no other complementary sequences to these probes. Buffers and solutions: LE consisted of 200 mM HCl and 400 mM BISTRIS (pH 6.5) and the TE consisted of 10 nM MES and 20 mM BISTRIS (pH 6.8). 1% 1.3 MDa polyvinylpyrrolidone was included in the LE to suppress electroosmotic flow. In experiments using PNA probes, the 30% v/v acetonitrile was included in the TE to maintain PNA solubility. All chemical stocks were obtained from Sigma-Aldrich Israel (Rehovot, Israel).

Additional ITP buffers used: LE was composed of 100 mM HCl, 200 mM BISTRIS (pH 6.5), and 1% (w/v) PVP; the latter was added to suppress electroosmotic flow. TE was composed of 50 mM MES and 100 mM BISTRIS (pH 6.8).

Premix preparation: At the beginning of every working day and before performing dilutions, the Morpholino stock solution was vortexed and heated it to 65° C. for 10 min (vortexing after 5 min). For each experiment, 30 μL of premix containing 100 nM Morpholino probes and the desired concentration of DNA were prepared, in TE. Premix of the control experiments contained only 100 nM Morpholino (no DNA), in TE. the premix was incubated at room temperature for 5 minutes before performing an experiment. It was found that use of a freshly thawed DNA aliquot every day was important for assay repeatability, and thawed DNA solutions were kept on ice during the experiments.

Washing steps: these washing steps were used before each experiment prior to filling the channels with LE: Rinse with bleach for 2 min, Rinse with 1 M NaOH for 2 min, Rinse with 1 M HCl for 2 min, Rinse with DI water for 2 min, and Rinse with LE for 2 min.

Experimental and optical setup: Experiments were performed on an inverted epifluorescent microscope (Ti-U, Nikon, Tokyo, Japan), using a commercially-available microfluidic chip (NS95x, PerkinElmer, Waltham, Mass., USA) placed on top of a transparent heating element (Cell MicroControls, Norfolk, Va.). All experiments used a 20× objective with NA=0.5 (Nikon Plan Fluor, Nikon Instruments, Melville, N.Y.), and images were recorded using a CCD camera (Clara, Andor, Belfast, UK) and analyzed using MATLAB (R2015a, Mathworks, Natick, Mass.) with lissamine-labeled morpholino probes used a custom designed filter cube with a 562/40 nm (FF01-562/40-25) excitation filter and a 630/69 nm (FF01-630/69-25) emission filter from Semrock (Rochester, N.Y., USA), and a 588 nm (T5881pxr) dichroic mirror from Chroma (Bellows Falls, Vt., USA), mounted on a Nikon TE2000/Ti cube purchased from Chroma. This filter cube was designed using the filter cube optimization code written in MATLAB (R2015a, Mathworks, Natick, Mass.).

### Assay Protocol

At the beginning of each set of experiments, a premix containing a large volume of the probes, targets, and TE at the desired concentrations was prepared for each experimental condition. In each experiment, 10 μL LE were added into the LE reservoir and the North and South reservoirs, and then applied vacuum from the TE reservoir to fill the channel with LE. After rinsing the TE reservoir with distilled water, 10 μL of the relevant premix were added into the TE reservoir. Using a high voltage sourcemeter (2410, Keithley Insturments, Cleveland, Ohio), +1000 V was continuously applied to the LE reservoir with the ground electrode at the TE reservoir, and simultaneously measured the resultant current. The sourcemeter was controlled and the current recorded using MATLAB (R2015a, Mathworks, Natick, Mass.). In between experiments using PNA probes, the channel was washed with bleach, 1 M NaOH, and 1 M HCl, then all reservoirs were rinsed with distilled water before LE was introduced. Between experiments with morpholinos, the wash step was omitted and the reservoirs were simply rinsed with distilled water before the introduction of the fresh LE.

After mixing the probes and targets together in the premixes, at least 15 minutes lapsed (1 hour in the case of 14-nt morpholinos) to allow the reaction to reach steady state before recording data. Recording data with much shorter incubation times yielded highly variable signals (data not shown). Except in the data presented in FIG. 4, the order of the experimental conditions was staggered to correct for potential time-dependent variations arising from the use of the same premixes over the course of many hours: for example, if working with conditions A, B, and C, experiments in the order ABC ABC ABC instead of AAA BBB CCC were performed to ensure that there were no significant differences in the incubation times for different conditions. No significant variation in signal with time post-mixing in any condition among the data collected was observed.

### Image and Data Analysis

Image analysis was performed in MATLAB (R2015a, Mathworks, Natick, Mass.). In all captured videos, the channel was aligned horizontally (such that image rotation was not necessary), and the top and bottom edges of the channel were selected manually and input into the code. Background signal was approximated by averaging the intensity of pixels in the channel in the first 15 frames of each video before the arrival of the ITP plug. Frames showing the ITP plug were automatically identified as those having a signal equal to at least the mean +1.75 standard deviations of the background, and the automatic selections were manually reviewed and corrected as necessary to ensure that all images of the ITP plug were included in the analysis. A flat-field correction image was generated by subtracting an image of the channel filled with water from an image of the channel filled with a fluorescent dye; this image was scaled by dividing each pixel intensity by the mean pixel intensity. The ITP images were then divided by the scaled flat-field correction image after background subtraction to perform the flat-field correction.

The intensity of the ITP plug in each image was obtained as follows: intensities of pixels within the channel (after background and flat field correction) were averaged along the width of the channel to generate a signal intensity profile along the length of the channel; this profile was smoothed with a 3-pixel wide moving average filter. In experiments performed with morpholino probes (FIGS. 6-9), the horizontal limits of the ITP plug were defined as those between which there was a smoothed intensity greater than the larger of two possible cutoff values: either 10% of the peak of the smoothed profile, or mean +3 standard deviations of the background signal (as calculated from frames that were 3 frames before and after the ITP plug). Signal was averaged from all pixels within the selected horizontal limits on the corrected image (not the smoothed profile). When working with ITP plugs of considerably different lengths, as in FIG. 9, signal was summed rather than averaged over the entire ITP plug. Intensities from all frames in which an ITP plug appeared were then averaged to yield the final intensity characterizing the experiment.

In videos in which no signal could be observed by eye, the code chose the image with the highest total pixel value within the channel to analyze. If an image yielded no signal that was above the threshold set by the mean +3 standard deviations of the background, the intensity was recorded as this threshold value with the note that the signal was below the threshold. Such “below threshold” or “signal not detected” intensities are shown as downward-facing triangles in FIGS. 6 to 9, and the horizontal line in these figures estimates this threshold across all experiments. In figures in which there was an undetectable “below threshold” signal that was higher than an “above threshold” detected signal, the threshold line was estimated at the value of the highest “below threshold” signal. In figures in which all “detectable” signals were higher than all “below threshold” signals, the line is shown halfway between the lowest “detectable” signal and the highest “below threshold” signal, but only if they were much less than an order of magnitude apart. In all figures, uncertainty bars represent the 95% confidence interval on the mean of all experiments performed at the same conditions; if an experimental condition yielded both “below threshold” and “detectable” values, they were averaged together.

Image analysis for experiments using PNA probes (FIGS. 4 and 5) were performed using an older version of the code (not included); the differences from the above description are that the horizontal limits of the ITP plug were rigidly defined as ±10 pixels to either side of the smoothed peak, and that the image yielding the highest intensity was taken to characterize the experiment instead of averaging intensities over multiple frames. In the experiments with E. coli-specific PNA probes (FIG. 4), when no signal could be detected by code or by eye, it was recorded as “below limit of detection” without quantification—these are shown as asterisks below the “Limit of detection” line in FIG. 4.

Experiments performed at room temperature using the metal halide light source typically captured 5-6 frames containing the ITP plug, whereas experiments at higher temperatures captured 3-5 frames due to increased ITP plug velocity at higher temperatures. ITP plugs appeared slightly wider at higher temperatures; this is most likely due to smearing of the image because of faster motion at higher temperatures, but could also be due to the temperature dependence of the width of the ITP interface. Experiments performed using the laser as a light source used a narrow field of view and a high frame rate (see Section 0), ˜7 frames including the ITP plug were captured in each experiment but only in ˜3-4 was the ITP plug directly on the laser spot; since in these experiments data was recorded from the frame yielding the highest signal (see above), this ensured that data was collected only from an image in which the plug was colocalized with the laser spot; this was also confirmed by manual examination of the selected images.

To enable comparison among experimental conditions with different widths of the ITP interface, experimental or modeled results as concentrations were not presented, but rather as total amounts, represented through the measured total intensity I=f·N, where f is an empirically-determined conversion factor from total measured intensity (I) to mole number (N).

In all experiments, a series of videos at 11 fixed locations (“stations”) were captured along the channel (FIG. 11). The last frame in which the complete plug was visible at each station was used as the data image, and a frame showing only LE solution before the ITP interface arrived at that station was used as the background image. Areas of the data image outside of the channel were cropped out, and background-corrected the result by subtracting the background image. To obtain the total intensity of the resulting data image, its pixel intensities were integrated, considering only pixels with an intensity higher than 2 standard deviations of the pixel intensities in the background image. No results are presented if no signal could be distinguished by eye from the video at a given station. For negative control experiments (with probes but without DNA), 11 images at the same stations at typical time points were captured and analyzed them by the same procedure. The quantitation threshold (horizontal dashed line in images) was set as the mean plus two standard deviations of signal from 3 repetitions of negative control experiments across all 11 locations at the relevant temperature.

Fitting to data: γ≡μ_{PD}/μ_{T}>1 was defined as the ratio of the mobility of the probe-NA hybrid μ_{PD} to that of the terminating ion in the TE zone μ_{T}. Using SPRESSO, with correction for ionic strength, were calculated μ_{T}=1.8·10⁻⁸ m²(V·s)⁻¹ for the current buffer system. The mobility of the probe-NA hybrid is not known precisely, but a reasonable upper bound is the mobility of free DNA, measured by Righetti et al. to be 3.7·10⁻⁸ m² (V·s)⁻¹ in Tris-acetate-EDTA buffer at 25° C. To be slightly conservative in terms of focusing rates, a value of γ=1.8, was used, throughout, as shown in FIG. 12, these results are essentially insensitive to changes.

Borosilicate glass microfluidic chips (model NS12A, see FIG. 11): were purchased from PerkinElmer (Hopkinson, Mass., USA). Polydimethylsiloxane (PDMS-SYLGARD 184 kit) was purchased from Polymer G (Kibbutz Gvulot, Israel) and attached PDMS-made reservoirs to the chip using oxygen plasma (corona treater, BD-20ACV Electro-Technic Products, Chicago, Ill., USA). Standardization of chip operating temperature: the temperature of the microfluidic chip was controlled using an indium tin oxide (ITO)-coated glass surface (Cell MicroControls, Norfolk, Va., USA), controlled by a temperature controller (Digital mTCII 2Ch micro-Temp controller, Norfolk, Va., USA). Due to observable mechanical stresses, the ITO-coated heating element was placed partway through the study. To check for consistency in temperature regulation between the old and new heating elements, a reference temperature T_{ref} was defined as the minimum temperature at which the 20/25 bp hybrid exhibits non-monotonic behavior and characterized T_{ref} using each heating element. It was found T_{ref} to be 45° C. when using the old heating element (as in FIGS. 8), and 53° C. when using the new heating element (FIGS. 9 and 10). Therefore, for standardization of experimental conditions, all temperatures are presented as relative to T_{ref} in the system used, instead of referring to the temperature displayed by the controller.

Preparation of chip reservoirs: a ˜5 mm thick PDMS layer was attached to the top surface of the reusable NS12A chip to increase the chip's reservoir volume. PDMS was prepared using a 10:1 (w/w) ratio of polymer to cross-linker, then casted the mixture into a flat dish, and cured it at 80° C. overnight. A rectangular PDMS piece of the size of the chip was cut and punched 5 mm holes at locations corresponding to the reservoirs. The inner surface of the PDMS and the top surface of the glass chip were exposed to oxygen plasma for 30 seconds each before being placed in contact; the resulting chip was then incubated at 80° C. for 2 hours.

Protocol for the accumulation-dissociation assay: the chip was placed on top of the ITO heating element (preheated to the desired temperature) on the microscope stage. The assay was performed immediately after washing the chip according to the above protocol. The LE was placed in the North, South and West reservoirs (FIG. 11) and filled the channel with LE by applying vacuum from the East reservoir for 2 min. The LE in the North was then replaced, South and West reservoirs with 30 μL of fresh LE, rinsed the East reservoir with DI water, and loaded 30 μL of the experimental premix (after a 5 min incubation at room temperature) into the East (TE) reservoir. The positive electrode was placed in the West reservoir and the ground electrode in the East (TE) reservoir and applied a constant current of 1 or 2 μA using the high voltage sourcemeter. The signal at 11 fixed locations (“stations”) along the channel (FIG. 11) was measured.

Number of hybrids at ITP interface: As provided hereinabove, the model predicts the time-dependent amount of hybrids (in moles) at the ITP interface to be in the general case N_{PD}^{ITP} (t)=γu_{ITP}Aηk_{off}⁻¹(e^{-k}^{_{off}}^{T/γ}−e^{-k}^{_{off}}^{t})c_{PD}^{w} and in the case of k_{off} and/or t sufficiently small that k_{off}t«γInγ/(γ−1), N_{PD}^{ITP} (t)=(γ−1)u_{ITP}Aηc_{PD}^{w}t.

Concentration of hybrids in TE reservoir: the concentration of probe-NA hybrids in the TE reservoir was assumed to be governed by a first-order reaction driven by the high concentration of probes, and has reached the steady-state value

$${c}_{P D}^{w} = \frac{{c}_{D}}{1 + \frac{{k}_{off}}{{k}_{on} {c}_{P}}}$$

where c_{D} and Cₚ=100 nM are the total concentrations of DNA and probes in the experimental mixture, respectively. It was assumed that c_{PD}^{w} is constant throughout the course of the experiment, despite the fact that the experiment begins at an elevated temperature immediately after the probe-DNA mixture is transferred from room temperature where k_{off}<<(kₒₙ c_{P}) and thus all DNA is hybridized (c_{PD}^{w}=c_{D}). In the case of the 4/25 bp mismatch, k_{off}>1>>kₒₙc_{P} for the experimental temperatures (see Section S12), making the time constant to reach a new steady state τ=(kₒₙc_{P}+k_{off})⁻¹<1 s; thus, c_{PD}^{w} reaches its steady state at the chip's temperature immediately. In the cases of the 25/25 by match and the 20/25 by mismatch, k_{off}/(kₒₙ c_{P})a 0.1, so 0.9 c_{D}<c_{PD}^{w}<c_{D} at the elevated temperature, and thus the difference between C_{PD}^{w} at room temperature and at the chip's temperature is negligible, enabling us to regard c_{PD}^{w} as constant.

Fitting linearly increasing data: For each experimental condition (operating temperature and applied current), the sets of linearly increasing experimental data that correspond to the 25/25 targets, or to hybridization at temperatures significantly below the melting temperature of the hybrid at that condition were fitted. It was assume for these cases that the DNA in the reservoir is fully hybridized and thus c_{PD}^{w}=c_{D}. Using the fitlm function of MATLAB (linear regression using ordinary least squares; version R2015a), the factor was obtained, where f represents the conversion from number of moles at the interface (N_{PD}^{ITP}) to the measured intensity. Using γ=1.8 as described, and the ITP interface speed u_{ITP} obtained for each experimental condition by measuring the time required for the plug to travel between equidistant imaging stations, the scale factor fAη was determined for each experimental condition, thus fixing all parameters in Equations other than kₒₙ and k_{off}.

Fitting non-monotonic data: For each set of non-monotonic data in FIGS. 8 and 10, k_{off} and kₒₙ were fitted iteratively. In each fitting, the maximum value of kₒₙ to 3·10⁵ s⁻¹ was restricted. In the first iteration, k_{off} was bound using an estimate of tₘₐₓ: the station with the highest average signal over three repeats was chosen, and the minimum and maximum possible tₘₐₓ values were used as the earliest and latest times recorded at the preceding and following stations, respectively. The values of tₘₐₓ were used to obtain the bounds on k_{off}. The fit function of MATLAB (nonlinear least squares method, trust region algorithm) was then used, and the above bounds for kₒₙ and k_{off}, fit to the data, to obtain a first approximation for kₒₙ and k_{off}. In subsequent iterations, k_{off} was bound based on the decrease of the signal below the quantification threshold: and k_{off} bounds was solved using as input the last fitted value of kₒₙ, the value of the quantitation threshold, and the times of the latest data point above the threshold and the earliest data point below it during the signal's decrease. The relevant equation was fit to the data as previously, but now using these new bounds on k_{off} and the last fitted value of kₒₙ to obtain a new value for k_{off}. Next, the fitting was repeated using the last fitted value of k_{off} to obtain a new fit for kₒₙ (within the kₒₙ bounds). Using this new value of kₒₙ, the bounds on k_{off} were reevaluated, and continue iterating k_{off} and kₒₙ fittings in this way until the last fitted value of k_{off} falls within the last-set bounds. No fitting was performed in the case of T=T_{ref}+15° C., as it has insufficient data for a reliable fit.

Model validity and onset of rehybridization: the model assumes that hybrids that dissociate at the ITP interface leave the interface so quickly that they are unable to rehybridize. However, rehybridization at the interface could occur if the concentration of available binding sites at the interface c_{bs}^{ITP} is sufficiently high that the characteristic times for hybridization τ_{hyb}=1/(cₛ^{ITP}·kₒₙ) becomes less than the timescale of free probe departure from the interface τₑₓᵢₜ=δ/u_{ITP}, for an ITP interface of width and a hybridization rate kₒₙ. This occurs when the concentration of available binding sites at the interface rises above the critical concentration c_{bs,crit}^{ITP}=u_{ITP}/(δ·kₒₙ), corresponding to a critical amount of N_{bs,crit}^{ITP}=u_{ITP}·A/kₒₙ. When the concentration of available binding sites at the interface reaches N_{bs.crit}^{ITP}, the equation is no longer relevant.

FIG. 13 presents an experimental characterization of the onset of rehybridization at the ITP interface, as a function of initial concentration and applied voltage, using 20/25 bp mismatches. As the number of available binding sites at the interface can be estimated by the total number of mismatches present (dashed curves), the time to reach N_{bs,crit}^{ITP} (solid horizontal line) can be estimated as t_{crit}=(kₒₙ(γ−1)ηc_{D}¹)⁻¹ (vertical lines). Rehybridization after t_{crit} prevents complete decay of the signal. As shown in FIG. 13, different currents yield different N_{bs,crit}^{ITP}, through dependence on u_{ITP}.

Available binding sites at the ITP interface: in addition to the 25-nt primary binding region, the 20/25 mismatch sequences also contain additional 4-nt sequences complementary to the probe, allowing a single mismatch sequence to bind to multiple probes simultaneously. Since 4-nt hybridization bonds are sufficiently weak that they dissociate after <150 s in the channel, most of the 20/25 mismatches reaching the ITP interface will have an available 4-nt binding site. Therefore, every mismatch at the interface contains at least one available binding site, regardless of whether its main 25-nt binding site is occupied, and thus the approximate number of available binding sites at the interface as the total amount of mismatches at the interface, is given by N_{D}^{ITP} (t)=(γ−1)u_{ITP}Aηc_{D}^{w}t. Setting this total number equal to the critical amount of moles N_{bs,crit}^{ITP}=u_{ITP}A/kₒₙ at which rehybridization begins, the critical time t_{crit}=(kₒₙ(γ−1)ηc_{D}^{w})⁻¹ was obtained when this amount is reached.

Estimation of off-rate values for NA-probe hybrids: to estimate k_{off} (e.g. in FIG. 4) for different NA-probe hybrids at different temperatures T, k_{off}=kₒₙ·K_{d}=kₒₙe^{(ΔG/(RT))} with kₒₙ=10⁵ Ms⁻¹, was used, Gibbs free energy of melting AG determined from DINAMelt, and R the universal gas constant. Morpholino-DNA hybrids are known to have a higher binding energy than the DNA-DNA binding energies provided by DINAMelt, so actual values of k_{off} in our experiments will differ.

TABLE 5
Thermodynamic parameters and off-rates k_{off} for binding of 25-nt sequences to probe. Calculated k_{off} in [Ms⁻¹],
ΔG in [kcal · mol⁻¹] from DINAMelt two-state melting (http://unafold.rna.albany.edu/?q=DINAMelt/Two-state-melting),
using [Na⁺] = 100M, [Na⁺] = 100 μM, [Mg⁺⁺] = 1 μM, strand concentration = 0.01 μM.

| | Bound bases: | | | | | | | | | |
|---|---|---|---|---|---|---|---|---|---|---|
| | 25/25 ^{[a]} | | 24/25 ^{[b]} | | 22/25 ^{[c]} | | 20/25 ^{[d]} | | 4/25 ^{[e]} | |
| Temperature [° C.] | ΔG | k_{off} | ΔG | k_{off} | ΔG | k_{off} | ΔG | k_{off} | ΔG | k_{off} |
| 55 | −18.5 | 4.7 · 10⁻⁸ | −15.7 | 3.5 ·10⁻⁶ | −13.3 | 1.4 · 10⁻⁴ | −12.6 | 4.0 · 10⁻ [symbol] | −1.6 | 8.6 · 10³ |
| 60 | −15.8 | 4.3 · 10− [symbol] | −13.1 | 2.5 · 10− [symbol] | −11.3 | 3.8 · 10− [symbol] | −10.7 | 9.5 · 10⁻³ | −1.3 | 1.4 · 10⁴ |
| 65 | −13.0 | 3.9 · 10⁻⁴ | −10.6 | 1.4 · 10⁻² | −9.3 | 9.7 · 10⁻² | −9.0 | 1.5 · 10⁻ [symbol] | −0.9 | 2.6 · 10⁴ |
| 70 | −10.2 | 3.2 · 10⁻² | −8.0 | 8.0 · 10⁻¹ | −7.4 | 1.9 | −7.4 | 1.9 | −0.6 | 4.1 · 10⁴ |

[symbol]

indicates data missing or illegible when filed

The below is also provided in FIG. 18.

(SEQ ID NO: 22)
[a] TGCTCATTGACGTTACCCGCAGAAG (target)
(SEQ ID NO: 23)
[b] TGCTTATTGACGTTACCCGCAGAAG (1-bp mismatch)
(SEQ ID NO: 24)
[c] TGCTGGATGACGTTACCCGCAGAAG (3-bp mismatch)
(SEQ ID NO: 25)
[d] TGCGACGCGACGTTACCCGCAGAAG (5-bp mismatch)
(SEQ ID NO: 26)
[e] TATACATTCCATAGATCTGGATACC (20-bp mismatch)

### Example 1

### The Morpholino Probe

A morpholino probe targeting the E. coli-specific 16S RNA sequence was designed. The probe was 25 nt long. The binding signal of the probe—DNA sequences was measures at various temperatures.

The following target sequences were used: a sequence with an entirely complementary region (M), a sequence with a region that was complementary except for a 5 nt mismatch in the middle, and a “random” sequence that had two 4-nt sequences that matched a 4-nt subsequence of the probe (R) (see

Table 3). As shown in FIG. 1, the 25-nt morpholino probe could distinguish between the matching sequence and the random sequence (2 orders of magnitude difference) at 20-60° C.

At the same time, it was not able to consistently distinguish between the matching sequence and the 5 nt-mismatch sequence: the only considerable difference in signal was obtained at 55° C., and it was only ˜1 order of magnitude. As temperature was increased above 55° C., the signal from all sequences decreased considerably, as would be expected from increased melting at higher temperatures.

### Example 2

### Shorter Morpholino Probe

Shorter probes are expected to be more specific regarding several-nucleotide-long mismatches. Thus, a shorter morpholino probe to the E. coli-specific 16S RNA sequence was designed using the same 14-nt sequence used for the PNA E. coil-specific probe (Table 1). The signal from the binding of this probe to a matching DNA sequence was measured. Additionally, the binding to DNA sequences containing 1-2 nt mismatches at the edge of the complementary sequence was measured. Finally, the binding of the probe to a “random” DNA sequence with two 4-nt-long matching subsequences, was measured, as described above.

As shown in FIG. 3, there was a ˜1.5-order of magnitude difference between the signal from the match and from the random sequence at room temperature (20° C.), and at higher temperatures, the signal from the random sequence was indistinguishable from the probe-only control.

At room temperature, the signal from the 1 nt mismatch was indistinguishable from that from the match, and the signal from the 2 nt mismatch was only slightly lower than the signal of the match. At 40° C., however, the difference in signal between the match and the 1-2 nt mismatches was larger than 1 order of magnitude.

Since the 25-nt probe was barely able to achieve a 1 order of magnitude difference between the signal from the match and the signal from a 5 nt mismatch (see FIG. 2), these results demonstrate that, indeed, a shorter probe is more specific to single-nucleotide mismatches than a longer probe of the same type. In fact, these results suggest that morpholinos may be more specific than PNAs, since the 14-nt PNA probe demonstrated considerably less then than an order of magnitude difference between the signal from the matching target and from the 1-2 nt mismatches at 40° C. However, this remains to be confirmed, since the 14 nt PNA probe was tested with mismatches in the center of the probe sequence and not at the edge of the probe sequence as in the case of the morpholinos, and this may have contributed to the observed difference. It can be seen that the 14 nt morpholino probe exhibits higher melting at 55° C. than the PNA probe does: the signal from the PNA probe decreases less than one order of magnitude between 20° C. and 55° C., whereas it decreases ˜2 orders of magnitude over this temperature range for morpholino probes of the same length.

### Example 3

### Lower Probe Concentration

Following the theoretical discussion that the signal to noise ratio (SNR) can be lowered by lowering the probe concentration, experiments with a lower probe concentration were performed. FIG. 8 shows the experimental results from the 25-nt morpholino probe binding to matching and random sequences, as described above, with the probe and DNA present at 1-10 nM. As can be seen, there is an almost 2 orders of magnitude drop in signal between the case of a 1 nM matching target with 10 nM of probe and with 1 nM of probe. This drop most likely comes from the additional time needed to reach equilibrium when there is a lower concentration of probes (see Section 2.1, Eqn. (2.6)). Since the resultant signal for the match at the lower concentration of probes is less than one order of magnitude above the threshold below which signals are not reliably detectable, operating at this concentration without giving the reaction more time does not allow for enough dynamic range to be able to detect a 10× lower concentration. Thus, unless one is willing to sacrifice more time for the reaction to reach equilibrium, it is best to continue working at 10 nM probe concentrations.

### Example 4

### Specificity to Random Binding

Finally, the response of the system to a concentration of “random” binding sequences higher than the concentration of probes was measured. 10 nM of the 25 nt morpholino probe was used, but, with 10 nM-10 μM of the “random” target, which contained two identical 4-nt subsequences that matched a region of the probe. As the results in FIG. 5 show, “random” DNA concentrations of 100 nM-10 μM all yield approximately the same signal.

Interestingly, the signal does not increase as the concentration of the random DNA increases past 100 nM, the probe is not saturated: the signal from the 10 nM Match DNA binding to the target remains one order of magnitude higher.

### Example 5

### Implementation of Multiple Measurements

Several practical options exist for implementing kinetic measurements in ITP. In one setting the ITP channel is equipped with two or more detectors along the path of the ITP. Since ITP is electromigrating at a velocity μ_{ITP}, it will transverse the different detectors at different time. The detectors should be located sufficiently apart to allow significant changes in signal to occur between stations, with characteristic distances between 5 mm and 5 cm.

By applying a flow velocity (e.g. pressure driven, electroosmotic) in the opposite direction to ITP electromigration, the ITP interface is held in place, precisely above the detector. The ITP process continues as usual, except it is stationary, allowing a single detector to be used in measuring the kinetic response (FIG. 7).

A hybrid that dissociate at the ITP interface instantly leaves the interface so that it can't rehybridize. Unbound NA of different species and/or already-bound NA that have additional available binding sites contribute to N_{bs}^{ITP}, The number of hybrids at the ITP interface over time, for different values of k_{off} due to either changing temperature (FIG. 8A) or a changing number of NA bases that can bind to the probe (FIG. 8B). In one embodiment, at sufficiently low dissociation rates (k_{off}→0), the signal increases linearly during the experimental timescale (signal is presented on a log scale), and as k_{off} increases (due to raised temperature or decreased probe-matching), dissociation causes a decrease in signal.

The increase in concentration starting at ˜700 s for the 5-basepair (bp) mismatch (20/25 bp) at T=T_{ref} suggests that the concentration of available binding sites at the interface has reached N_{bs,crit}^{ITP} and thus dissociated probes are beginning to rehybridize at the ITP interface.

The linear increase in signal for targets vs. the exponential decrease in signal for mismatches during experimental timescales provides an opportunity for highly specific detection, even when mismatches are present at a much higher concentration.

As shown in FIG. 9 the signal vs. time obtained from 1 nM target and 10 nM mismatch (20/25 bp). After 500 s, the signal of the mismatch has decayed below the quantitation threshold, while the signal from the lower-concentration target continues to increase. At this operating temperature, further reducing the concentration of the target resulted in signals below the quantitation threshold, whereas increasing the concentration of the mismatch resulted in an amount of hybrids close to N_{bs,crit}^{ITP}. In order to demonstrate higher concentration ratios (FIG. 9B), the temperature was lowered and used a higher degree of mismatch (4/25 bp), resulting in clear distinction between target and mismatch at a 1:1000 concentration ratio.

As shown in FIG. 10 a mixture of targets (25/25) and mismatches (22/25) at concentration ratios of 1:10 to 1:1000 (match:mismatch), simulating cases of detection of a desired target in the presence of a high concentration of non-targets was analyzed. The black solid curves show the signal resulting from the mixture whereas the gray solid curves show the signal in the absence of a target (i.e. negative control—high concentration of non-targets).

In FIG. 10A, the case of the assay operated at 60° C., resulting in k_{off} values of 4.3·10⁻⁶ Ms⁻¹ and 3.8·10⁻³ Ms⁻¹ for the target and mismatch respectively (based on DINAMelt calculations) is presented. At early times, the signal of the mixture is dominated by the excess mismatches, and is undistinguishable from that of the negative control. However, at later times, the signal of the negative control decreases, as it is dominated by exponential dissociation, whereas the signal of the mixture begins to increase, as it becomes dominated by the (near linear) accumulation of the targets.

For sufficiently long times, the signal of the mixture merges with that of the target only (gray dashed line). The square symbols and dashed vertical lines indicate the earliest time at which the signal of the mixture containing the target exceeds that of the negative control by a factor of 3. From that point onward, detecting the target becomes increasingly easier, as the negative control signal continues to decay and eventually falls below the quantitation threshold (set here at an arbitrary value of 70, for demonstration purposes only), whereas the signal from the target continues increasing. As expected, as the ratio of mismatches to targets increases, the time required for detection of the target also increases. However, the time for detection exhibits a logarithmic dependence on the ratio of concentrations, i.e. detection of the target in a 1:1000 concentration ratio requires only approximately 3-fold longer than in a 1:10 ratio.

As shown in FIG. 10B, the time required to achieve a 3-fold difference in signal between the mixture and the negative control can be significantly reduced by operating at 65° C., where the k_{off} values of the target and mismatches are 3.9·10⁻⁴ Ms⁻¹ and 9.7·10⁻² Ms⁻¹, respectively. For example, for a 1:1000 ratio, this time is reduced from 50 min at 60° C. to 2 min at 65° C. However, as illustrated in FIG. 10, at such short times the signals may still be below the quantitation threshold, and a longer duration may be required before detection can be achieved (here at ˜4 min). Accordingly, reducing the quantitation threshold of the detection system would enable higher specificity at shorter times.

These results are generalized in the contour map of FIG. 10C, which shows how the time for target detection (x-axis) changes with the dissociation rate of the mismatch (y-axis), for different target:mismatch ratios (contour lines). For example, the two cases in FIGS. 10A,B are represented by horizontal dashed lines crossing the y-axis at their respective dissociation constant. These lines intersect the concentration ratio contours at different times, corresponding to the times indicated in FIGS. 10A,B. The quantitation threshold takes here the form of a minimum time for detection, before which the signal of even the target itself is too low to detect. As an additional example, it was shown the case of detecting a specific target in a mixture containing a high concentration of single-bp mismatches (24/25). The k_{off} of the mismatch at 65° C. is approximately 1.4.10⁻² and it would yield a 1:1000 specificity at approximately 15 min.

In summary, it is demonstrated that using non-focusing probes in continuous-injection ITP yields unique accumulation-dissociation dynamics, allowing for discrimination between targets and mismatches at high specificity. The main advantage of this assay is that it enables a gain in signal due to ITP focusing, while at the same time dissociating any non-specific hybrids. This is done in a single-step homogenous assay, avoiding any surface reactions or additional separation matrices. It is demonstrated that for a given analysis time, specificity is limited by the sensitivity of the system. Our standard CCD-based optical system showed a limit of detection of ˜100 μM, allowing demonstration of 1:1000 specificity for 4/25 bp sequences, and 1:10 specificity for 20/25 bp sequences.

1. A method for sequence-specifically detecting and/or separating a nucleic acid molecule, comprising the steps of:
a. contacting a mixture of nucleic acid molecules with a morpholino having a sequence of interest in a first solution and obtaining a nucleic acid molecule/morpholino hybrid;
b. introducing said first solution comprising said hybrid resulting from step (a) into an ITP system, said ITP system comprises a second solution of high effective mobility leading electrolyte (LE) ions and a third solution of low effective mobility trailing electrolyte (TE);
c. applying an electric field across said second solution and said third solution;
wherein said hybrid but not free morpholino focus at the sharp LE-TE interface in said ITP system, wherein said TE has a higher mobility than the morpholino probe and said TE has a lower mobility than the hybrid, thereby sequence-specifically separating a nucleic acid molecule.

2. The method of claim 1, wherein said sequence-specifically detecting is detecting a 1 to 5 nucleotides mismatch.

3. The method of claim 1, wherein said second or said third solution comprises said nucleic acid, said labeled morpholino or both.

4. The method of claim 1, further comprising a counterflow for maintaining said ITP interface, stationary.

5. The method of claim 1, wherein said detecting is achieved by using a photodetector, a photomultiplier tube (PMT), a conductivity detector, a radioactive detector, a camera or any combination thereof.

6. The method of claim 1, wherein said hybrid comprises a nucleic acid molecule having 10 or more nucleotides.

7. The method of claim 1, wherein said morpholino does not focus under ITP unless hybridized to said nucleic acid molecule.

8. A system comprising:
(a) A first zone comprising a solution of high effective mobility leading electrolyte (LE) ions;
(b) A second zone comprising a solution of low effective mobility trailing electrolyte (TE) ions;
(c) An anode and a cathode; and
(d) At least one detector situated between said anode and said cathode configured to provide at least two readings; or at least two detectors situated between said anode and said cathode.

9. The system of claim 8, wherein said detector or said detectors is a photodetector, a photomultiplier tube (PMT), a conductivity detector, a radioactive detector, a camera or any combination thereof.

10. The system of anyone of claim 8, further comprising a thermometer, a heating means, counterflow means or any combination thereof.

11. (canceled)

12. A method for separating a match hybrid comprising a first molecule and a second molecule, comprising the steps of:
a. contacting a mixture of molecules comprising said first molecule and said second molecule in a first solution and obtaining hybrids;
b. introducing the first solution into the ITP system, the ITP system comprises a second solution of high effective mobility leading electrolyte (LE) ions and a third solution of low effective mobility trailing electrolyte (TE);
c. applying voltage between the LE and the TE, inducing a low electrical field in the leading electrolyte and a high electrical field in the terminating electrolyte;
wherein only: (A) the hybrids; and/or (B) the hybrids and first molecule focus at the sharp LE-TE interface in the ITP system, thereby separating a specific hybrid.

13. The method of claim 11, wherein said first molecule and said second molecule are identical and said hybrid comprises a dimer.

14. The method of claim 11, wherein said first molecule is a protein said second molecule is a specific ligand of said protein; or said first molecule comprises an epitope recognizable by an antibody said second molecule is an antibody or a binding fragment of an antibody.

15. The method of claim 11, further comprising step (d) of detecting, wherein said detecting is achieved by using a photodetector, a photomultiplier tube (PMT), a conductivity detector, a radioactive detector, a camera or any combination thereof.

16. (canceled)

17. The method of claim 11, wherein said hybrids comprise a match hybrid and a mismatch hybrid.

18. The method of claim 14, wherein step (d) provides a measure for mismatch hybrid or is used to obtain a measure of false positive detection of a mismatch hybrid.

19-26. (canceled)
